(19) United States
(12) Patent Application Publication
(10) Pub. No.: US 2018/0326112 A1
(43) Pub. Date: Nov. 15, 2018

(54) FRAGRANCE DISPENSER AND SYSTEM, AND METHOD FOR USING THE SAME

(71) Applicant: SAVVY INC., Toronto (CA)

(72) Inventors: Muhammad Muneeb MUSHTAQ, Mississauga (CA); John BYARD, Burlington (CA); Muhammad Nabeel MUSHTAQ, Mississauga (CA); Jonathan Paul LOUDON, Toronto (CA); John Paul SCHMIDER, Toronto (CA); Rebecca Rosemary BRUNETTE, Toronto (CA); Terry Dean POPIK, Toronto (CA); Kevin TALLEVI, Brampton (CA)

(21) Appl. No.: 15/774,894

(22) PCT Filed: Nov. 14, 2016
(86) PCT No.: PCT/CA2016/051316

§ 371 Date: May 9, 2018

Related U.S. Application Data
(60) Provisional application No. 62/255,066, filed on Nov. 13, 2015.

(51) Int. Cl. A61L 9/14; H04L 12/58

(52) U.S. Cl. CPC A61L 9/14; H04L 51/046; G06F 9/44505; A61L 2209/111; A61L 2209/133; A61L 2209/132

(57) ABSTRACT

A fragrance dispenser, system and method are provided. The fragrance dispenser comprises a fragrance reservoir disposed in fluid communication upon an atomizer assembly. The atomizer assembly has an atomizer to permit emitting of liquid fragrance communicated from the fragrance reservoir toward the atomizer by gravity when the fragrance dispenser is in an operating orientation. The system includes a fragrance dispenser and at least one computer. The at least one computer can include a personal computer of the user and a remove computer. The system can, for example, issue commands to the fragrance dispenser or manage the use of a fragrance. The methods include, for example, a method of dispensing a fragrance and a method of managing the supply of a liquid fragrance is described.

### RELATED APPLICATIONS

This application claims priority from, or the benefit of, U.S. provisional application No. 62/255,066 filed on Nov. 13, 2015. U.S. provisional application No. 62/255,066 is incorporated herein by reference.

### TECHNICAL FIELD

The following relates generally to fragrance dispensers. In particular, the following relates to a fragrance dispenser and system, and a method for using the same.

### BACKGROUND

Fragrances, alternatively called aromas or scents, are volatile substances that produce a smell response when inhaled through the nose. Fragrances are used to enhance physical or mental well-being, or simply to make a space more enjoyable to be in. Many fragrances are synthetic, formulated to be both fragrant and easily manufactured or used. Other fragrances are bio-based. In particular, essential oils are hydrophobic liquids extracted from plants that contain the “essence” of the fragrance of the plant. Essential oils can be dispersed in the air with a nebulizer (alternatively called an atomizer) or by heating or burning them.

Ultrasonic wave nebulizers have a vibrating piezoelectric element in contact with a liquid. U.S. Pat. No. 3,738,574 describes an atomizer having a piezoelectric oscillator system. The system includes a piezoelectric transducer that vibrates a solid plate located above a liquid reservoir. A pump delivers liquid to the front of the plate. Excess liquid that is not atomized runs off of the plate. U.S. Pat. No. 4,301,093 describes a similar system wherein the liquid is brought up to the front of the plate by a wick rather than a pump. U.S. Pat. No. 5,297,734 describes an atomizer with a porous vibrating plate. Liquid is supplied to the back of the plate and a fog is produced from the front of the plate. Liquid delivery methods include releasing liquid from a reservoir above the plate at a controlled rate onto a fixed plate behind the vibrating plate, immersing the edges of the fixed and vibrating plates into the free surface of a liquid, using a wick to draw liquid upwards to a horizontal vibrating plate, and placing the vibrating plate horizontally just above the free surface of a liquid.

The patents mentioned above relate to nebulizers generally. S. C. Johnson & Son, Inc. has produced fragrance dispensers using a piezoelectric element with a vertical wick to draw the fragrance to the back of a horizontal porous plate. Vibrating the plate with a piezoelectric actuator sends droplets of liquid upwards from the front of the plate. These devices are described in, for example, U.S. Pat. Nos. 6,857,580 and 6,896,193. U.S. Pat. No. 7,610,118 describes a system with multiple atomizer assemblies and a microcontroller to control the emission of liquids from the atomizers according to one or more set programs.

### SUMMARY

This specification describes a fragrance dispenser and system, and methods for using the same.

The fragrance dispenser has a fragrance reservoir disposed in fluid communication upon a dispenser, the dispenser having an atomizer sealingly disposed thereon to permit emitting of liquid fragrance communicated from the fragrance reservoir toward the atomizer by gravity when the fragrance dispenser is in an operating orientation.

In the examples illustrated, the atomizer has a porous plate oriented at a slant to spray the fragrance upwards and horizontally away from the front of the porous plate. At most times (i.e. when the fragrance reservoir is not empty) there is a continuous volume of fragrance liquid extending from a free surface in the reservoir to the back of the porous plate. The free surface is located above the porous plate. An atomizer reservoir acts as a simple conduit carrying the fragrance liquid from the reservoir to the porous plate without an intervening device such as a valve or wick. By avoiding the use of a wick, we also avoid the need to replace the wick when replacing the fragrance reservoir, or mixing one scent with another if a reservoir with a new fragrance is used and the wick is not replaced.

The reservoir may be vented or not. Surprisingly, an excessive vacuum is not created in an unvented dispenser, possibly because some air enters through the porous plate when a burst of fragrance is dispensed. But despite this possibility, an unvented dispenser still inhibits unwanted liquid from accumulating on the front of the porous plate between bursts. Optionally, a piece of absorbent material can be placed on the front of the porous plate to temporarily collect any liquid fragrance on the front of the porous plate until the atomizer is operated again.

This specification also describes a fragrance system. The system can be used with a fragrance dispenser having gravity assisted flow as described herein or with another type of electrically operable fragrance dispenser. The system includes a fragrance dispenser and at least one computer physically separate from the fragrance dispenser. This computer is operated by the fragrance dispenser user and may be, for example, a smartphone. Optionally, the system includes a computer operated by the fragrance dispenser user and a remote computer. The computer or computers may be used to facilitate one or more functions such as causing the fragrance dispenser to emit a fragrance, indicating when a fragrance dispenser is empty or nearly empty, or storing information relating to the operation of the system. Optionally, the system may include multiple fragrance dispensers operated by one user.

Methods described herein can be used with the dispenser or system described herein or with other dispensers or systems. The methods include methods of dispensing a fragrance and methods of managing the supply of fragrance.

In the examples illustrated, a fragrance dispenser dispenses a fragrance in bursts. The fragrance dispenser may emit a single burst in response to a command, or a series of bursts in response to a command to emit fragrance over a period of time. A person can cause the fragrance dispenser to emit a burst of fragrance by pressing a button on the fragrance dispenser. Alternatively, the person can cause the fragrance dispenser to emit a burst of fragrance by pushing a button on their computer. In another alternative, the person can cause the fragrance dispenser to emit bursts of fragrance according to a scheduling program run on their computer or the remote computer. In other alternatives, an instruction to emit a burst can be created, delayed or cancelled in response to a signal from a motion detector. The bursts can also be counted to indicate when a reservoir is empty. The count can be maintained by any of the fragrance dispenser, the user's computer or the remote computer.

These and other aspects are contemplated and described herein. It will be appreciated that the foregoing summary sets out some aspects of a fragrance dispenser, system and method to assist skilled readers in understanding the following detailed description.

### BRIEF DESCRIPTION OF THE DRAWINGS

A greater understanding of the embodiments will be had with reference to the Figures, in which:

FIG. 1 is a front perspective view of a fragrance dispenser in accordance with one embodiment thereof;

FIG. 2 is a rear perspective view of the fragrance dispenser of FIG. 1;

FIG. 3 shows the fragrance dispenser of FIG. 1 with a front cover thereof having been opened;

FIG. 4 is a perspective view of a fragrance cartridge for use in the fragrance dispenser of FIG. 1;

FIG. 5 is a perspective view of a fragrance reservoir of the fragrance cartridge of FIG. 4;

FIG. 6 is a perspective view of a petal valve and a petal valve ring that are secured to the bottom of a fragrance reservoir tank of the fragrance reservoir of FIG. 5;

FIG. 7 is a perspective view of an atomizer reservoir forming part of an atomizer assembly of the fragrance cartridge of FIG. 4;

FIG. 8 is an exploded view of an atomizer disc and a wicking pad positioned adjacent an atomizer disc aperture of the atomizer reservoir of FIG. 7;

FIG. 9 is an exploded front view of the fragrance cartridge of FIG. 4 including the atomizer reservoir of FIG. 7 assembled in an atomizer assembly and the fragrance reservoir of FIG. 5;

FIG. 10 is a sectional view of the fragrance reservoir of FIG. 5 and the atomizer reservoir of FIG. 7 when assembled in the fragrance cartridge of FIG. 4;

FIG. 11 is a front perspective view of the fragrance dispenser of FIG. 1 with the front cover removed and having one fragrance cartridge as shown in FIG. 4 with a portion of the atomizer assembly removed installed therein;

FIG. 12 is a perspective view of a cartridge deck of the fragrance dispenser of FIG. 1;

FIG. 13 is a perspective view of a circuit board forming part of the base portion of the fragrance dispenser of FIG. 1;

FIG. 14 is a perspective view of a base cover forming part of the base portion of the fragrance dispenser of FIG. 1;

FIG. 15 is a front perspective view of a contact plate of the fragrance cartridge of FIG. 4 and a contact block of the circuit board of FIG. 13;

FIG. 16 is a side sectional view of the fragrance dispenser of FIG. 1 showing the cartridge deck of FIG. 12, the circuit board of FIG. 13, and the base cover of FIG. 14 assembled together;

FIG. 17 is a side view of the power adapter of the fragrance dispenser of FIG. 1;

FIG. 18 is a front perspective view of the power adapter of FIG. 17;

FIG. 19 is a rear perspective view of the power adapter of FIG. 17;

FIG. 20 is a rear perspective view of the fragrance dispenser of FIG. 2 with the power adapter thereof removed;

FIG. 21 shows a USB cable for connecting the power adapter of FIG. 17 to the main body of the fragrance dispenser of FIG. 20;

FIG. 22 shows a fragrance dispenser system utilizing the fragrance dispenser of FIG. 1;

FIG. 23 shows a method of initializing the fragrance dispenser system of FIG. 22;

FIG. 24 shows a method of ordering a replacement fragrance reservoir for the fragrance dispenser system of FIG. 22;

FIG. 25A shows an alternative fragrance dispenser;

FIG. 25B is an exploded view of the fragrance dispenser of FIG. 25A;

FIG. 25C is an exploded view of the base of the fragrance dispenser of FIG. 25A;

FIG. 25D is an exploded view of an atomizer assembly of the fragrance dispenser of FIG. 25A;

FIG. 25E is a cross section of a fragrance cartridge of the fragrance dispenser of FIG. 25A;

FIG. 25F is an exploded view of a power adapter of the fragrance dispenser of FIG. 25A;

FIG. 26 shows a sectional view of a fragrance cartridge in accordance with an alternative embodiment;

FIG. 27 is a schematic drawing of an alternative system;

FIG. 28 shows a process for initial set up of a dispenser and smartphone of the system of FIG. 27;

FIG. 29 shows a process to load a fragrance cartridge 48 into the dispenser in the system of FIG. 27;

FIG. 30 shows a method of managing fragrance use in the system of FIG. 27;

FIG. 31 shows a method for emitting a burst using the smartphone in the system of FIG. 27;

FIG. 32 shows a method of programming scheduled operation of the dispenser in the system of FIG. 27;

FIG. 33 shows a method of configuring a burst button of the dispenser in the system of FIG. 27; and,

FIG. 34 shows a method of configuring a motion sensor of the dispenser in the system of FIG. 27.

### DETAILED DESCRIPTION

For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practised without these specific details. In other instances, well-known methods, procedures and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

Any module, unit, component, server, computer, terminal, engine or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors.

The following provides a fragrance dispenser and system, and method for using the same. The described fragrance dispenser comprises a reservoir fillable with a liquid fragrance, and an atomizer assembly comprising an atomizer. The reservoir provides the liquid fragrance to the atomizer assembly, which selectively dispenses the liquid fragrance in accordance with a configurable dispensing schedule or other inputs by selectively activating the atomizer. In the described fragrance dispenser, the reservoir provides the liquid fragrance to the atomizer assembly at least in part by gravitational force. In other words, in an operating orientation, the fragrance reservoir is located at least partially above the atomizer assembly. A porous plate of the atomizer is slanted, for example at 30 to 60 degrees from horizontal. The system can be used with a fragrance dispenser as described herein or with other fragrance dispensers. The system includes a fragrance dispenser and at least one computer. Methods described herein can be used with the system described herein or other systems. In some examples, a method of dispensing a fragrance is described. In other examples, a method of managing the supply of a liquid fragrance is described.

A fragrance dispenser, to be described further below, dispenses a fragrance into a room. For example, a liquid fragrance may be dispensed by atomization such that droplets of the liquid are sprayed into the room. As used herein the word atomize, and its variants such as atomizing and atomization, include making an aerosol or a spray, mist, vapor, fog, cloud or another form of the liquid which appears to be atomized. An atomizer is a device suitable for atomizing a liquid such as an ultrasonic vibrating plate or a jet nebulizer.

The liquid fragrance may be a solution or emulsion of a fragrant compound in a carrier. The fragrant compound may be, for example, an essential oil or a blend of essential oils. The carrier may be water, an alcohol such as ethanol, or a mixture of water and alcohol. The liquid fragrance may also have one or more other additives, for example a surfactant, a detergent, an emulsifier or a denaturing additive. In one example, the liquid fragrance may comprise a mixture of ethanol, one or more essential oils, and optionally one or more additives. The ethanol may be present at 50% by volume or more or 60% by volume or more. The essential oil or oils may be present at 10% by volume or more or 20% by volume or more. The additive or additives may be present at up to 10% by volume. Using a large amount of ethanol (i.e. 50% by volume or more or 60% by volume or more) with one or more essential oils produces a low viscosity mixture which allows for a low power (i.e. 10 V at 2 A or less, or 5 V at 1 A or less) atomizer to be used. A large amount of ethanol also appears to create an aerosol, or at least a fine suspension, wherein the essential oils dissipate into the air rather than settling as droplets near the atomizer.

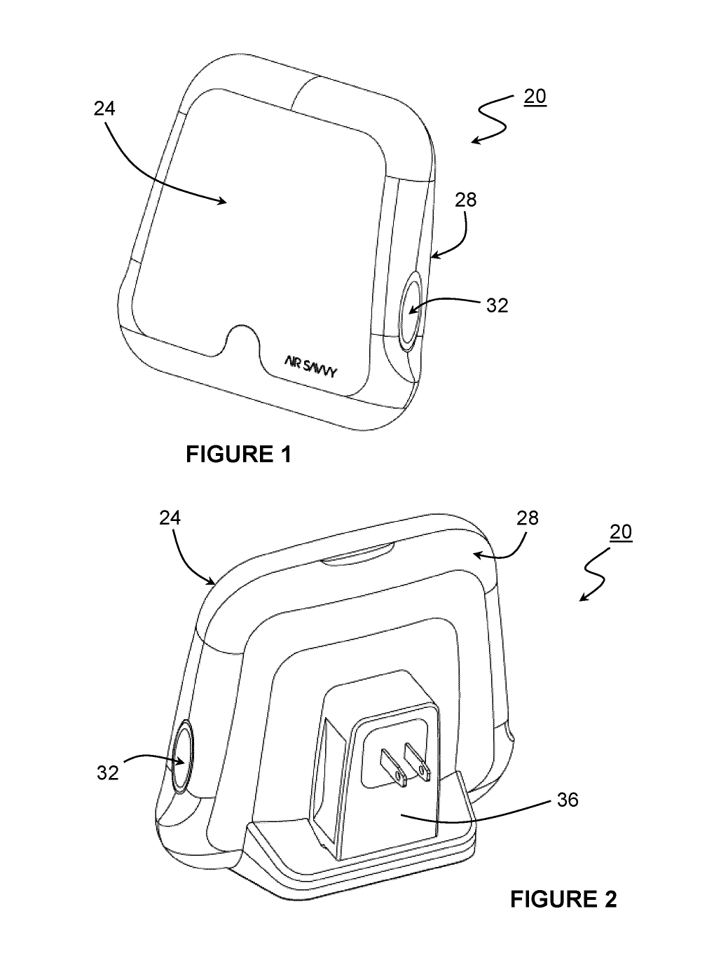

An exemplary fragrance dispenser 20 is shown in FIGS. 1 and 2. The fragrance dispenser 20 includes a front cover 24 removably coupled to a back cover 28, and a chimney 32, located at each side and preferably having an aperture substantially flush with an aperture created by the mating of the front cover 24 and the back cover 28. A power adapter 36 is removably coupled to the back cover 28 and enables the fragrance dispenser 20 to be plugged into a standard 115 or 230 volt alternating current (AC) electrical outlet to power the fragrance dispenser 20. The power adapter converts the AC power into low, i.e. 10 V or less or 5 V or less, direct current (DC) power.

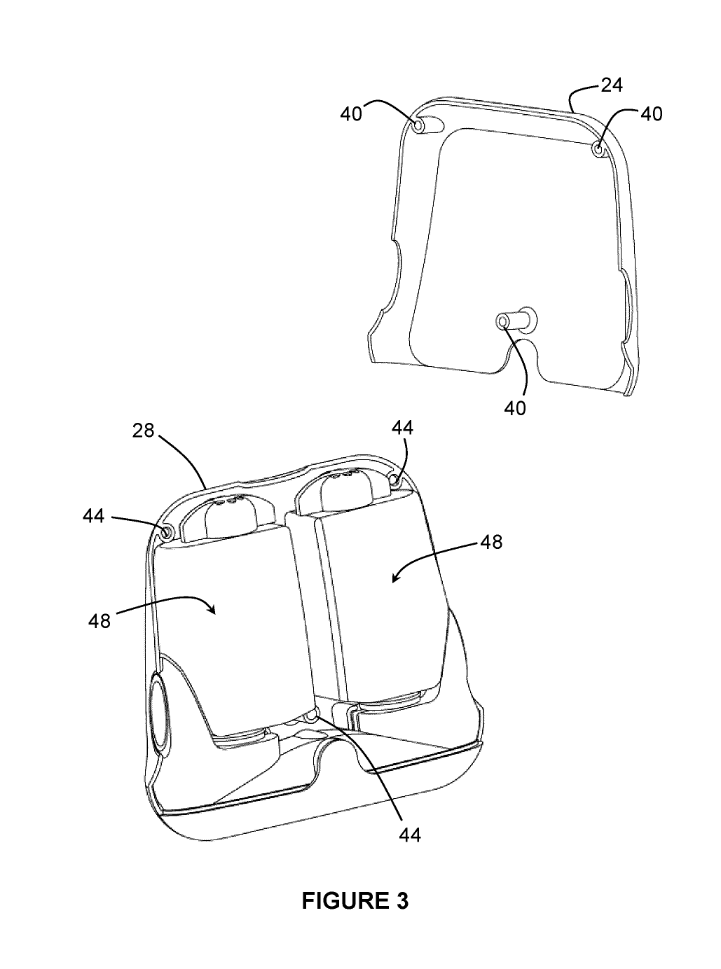

FIG. 3 shows the fragrance dispenser 20 with the front cover 24 removed. The front cover 24 includes a set of three bosses 40 that correspond to three bosses 44 of the back cover 28. Each of the bosses 40 of the front cover 24 has a ferromagnetic slug affixed therein and extending slightly out of bosses 40. Correspondingly, each of the bosses 44 of the back cover 28 has a magnet affixed at a slightly recessed position therein. The attraction between the magnets in the bosses 44 of the back cover 28 and the ferromagnetic slugs in the bosses 40 of the front cover 24 attracts the ferromagnetic slugs of the bosses 40 of the front cover 24 into the bosses 44 of the back cover 28 to hold the front cover 24 to the back cover 28. The front cover 24 can be manually separated from the back cover 28 via an application of a minimum separation force. It will be appreciated that the locations of the magnets and the ferromagnetic slugs could be reversed.

A pair of fragrance cartridges 48 is housed in the fragrance dispenser 20. Alternate embodiments contemplate one or a plurality of fragrance cartridges 48 housed, or houseable, in the fragrance dispenser 20. Where one such cartridge can be included, the fragrance dispenser 20 is limited to the dispensing of only the fragrance of the included fragrance cartridge 48; however, where a plurality of fragrance cartridges 48 are included, each fragrance cartridge 48 could be loaded with differing liquid fragrances, and such liquid fragrances could become known to the fragrance dispenser 20 (or an operator thereof), resulting in the advanced operation of a more intelligent fragrance dispenser that is capable of selectively dispensing any one or more of the included liquid fragrances.

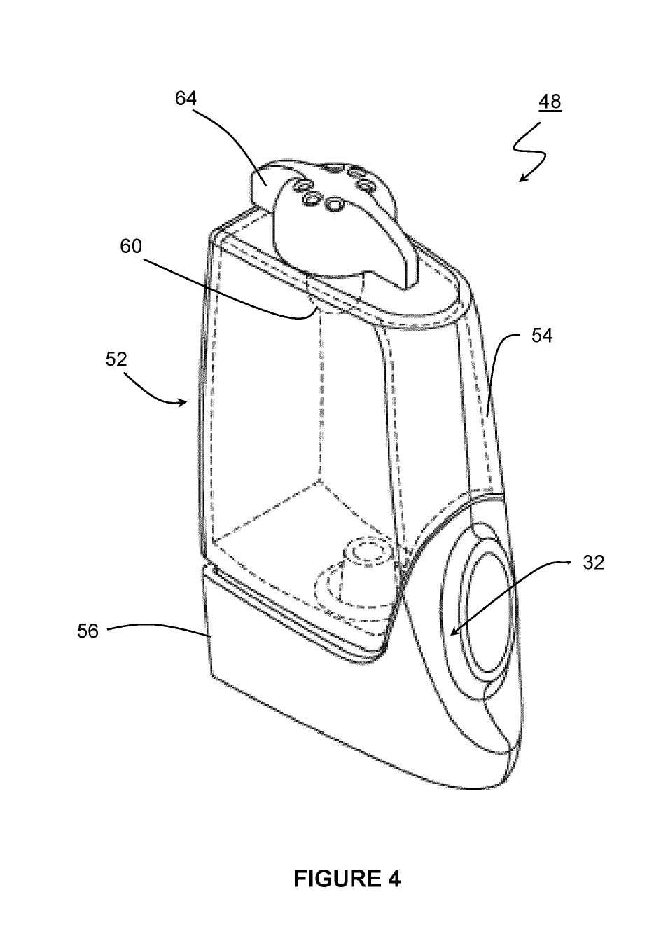

FIG. 4 shows one of the fragrance cartridges 48 in isolation. The fragrance cartridge 48 includes a fragrance reservoir 52 mounted atop an atomizer assembly 56. In various embodiments, the fragrance reservoir 52 may be affixed to the atomizer assembly 56 and treated essentially as a unitary piece. In various other embodiments, the fragrance reservoir 52 and the atomizer assembly 56 may be separate pieces, configurations for achieving which are described below.

The fragrance reservoir 52 has a fragrance reservoir tank 54 that is made from plastic such as clear polyethylene terephthalate (“PET”) that is formed, for example blow molded, and holds a liquid fragrance, such as a mixture of on re more essential oils and a carrier, to be atomized. Optionally, a valve is disposed at the top of the fragrance reservoir 52 to permit the entry of ambient air into the fragrance reservoir tank 54. The valve shown is embodied as a ball valve 60. Optionally, a sealing cap 64 is secured onto the top of fragrance reservoir tank 54.

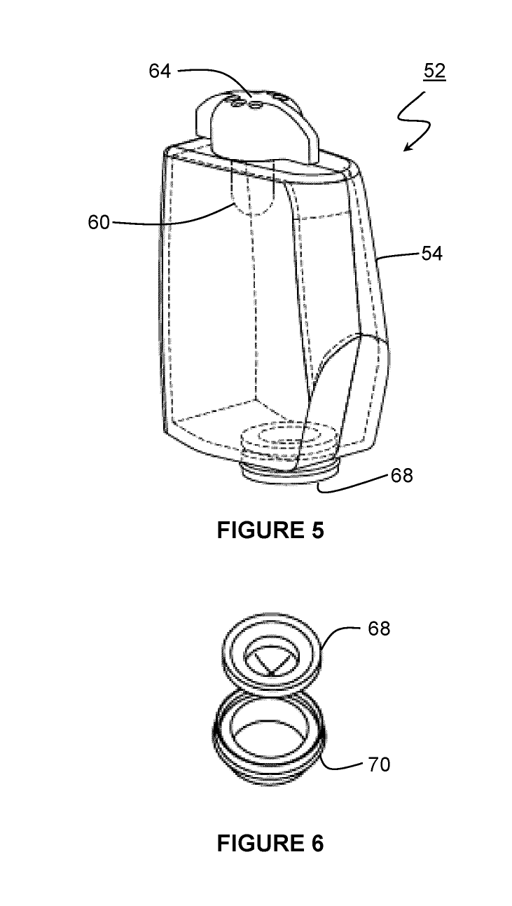

The fragrance reservoir 52 is shown separate from the atomizer assembly 56 in FIG. 5. On its bottom surface is an aperture with a ridged flange to which an optional petal valve 68 is secured via a petal valve ring 70 as shown in FIG. 6 that snaps onto the ridged flange of the fragrance reservoir tank 54. The petal valve 68 maintains a liquid-tight seal but can be opened via penetration by a solid object. Preferably, during transport and storage, a removable seal or cap is affixed to the petal valve 68 to prevent leakage.

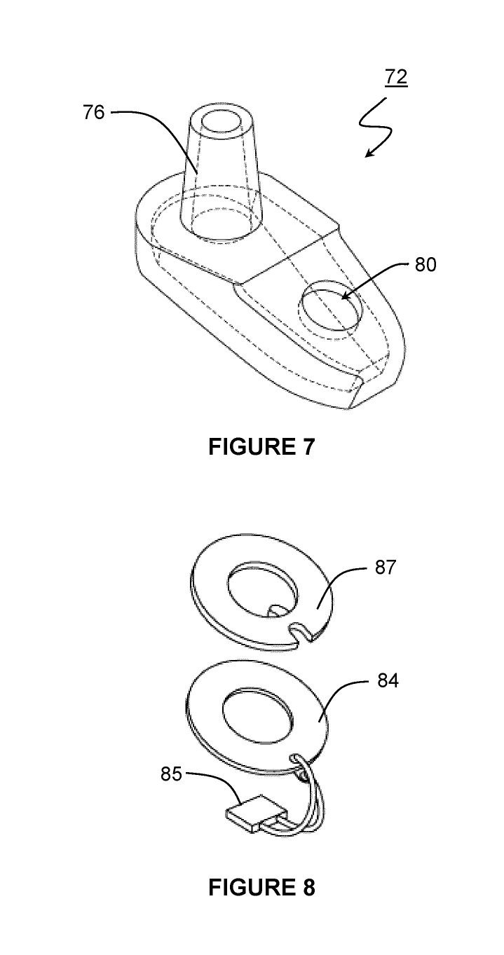

Within the atomizer assembly 56 is an atomizer reservoir 72 as illustrated in FIG. 7. The atomizer reservoir 72 has a spigot 76 at an upper end and an atomizer disc aperture 80 on an upper surface at a lower end. The spigot 76 mates with the petal valve 68 of the fragrance reservoir 52 to form a liquid-tight seal and permits liquid fragrance in the fragrance reservoir 52 to travel through the atomizer reservoir 72 to the atomizer disc aperture 80.

FIG. 8 shows an atomizer disc 84 that is sealingly secured to cover the atomizer disc aperture 80. Any suitable microporous atomizer can be employed, such as a 20-millimeter ceramic atomizer produced by Cosson. A contact plate 85 is coupled to the atomizer disc 84 via wiring and is secured to the undersurface of the atomizer assembly 56 to provide an electrical interface for powering the atomizer disc 84. The atomizer disc 84 uses ultrasonic vibration to generate and release a mist from the liquid fragrance in the atomizer reservoir 72 by breaking the surface tension of the liquid fragrance. A wicking pad 87 is disposed atop of the atomizer disc 84 to prevent liquid fragrance from pooling on the surface of the atomizer disc 84.

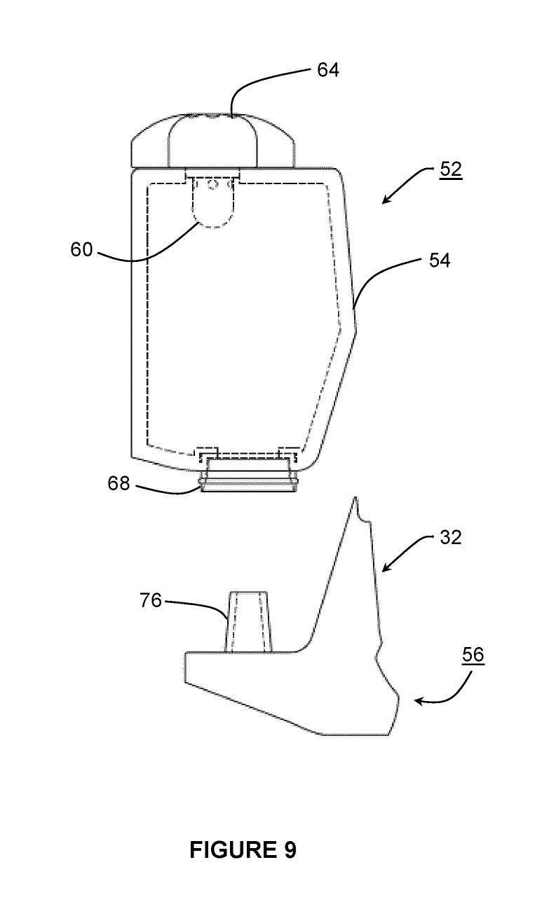

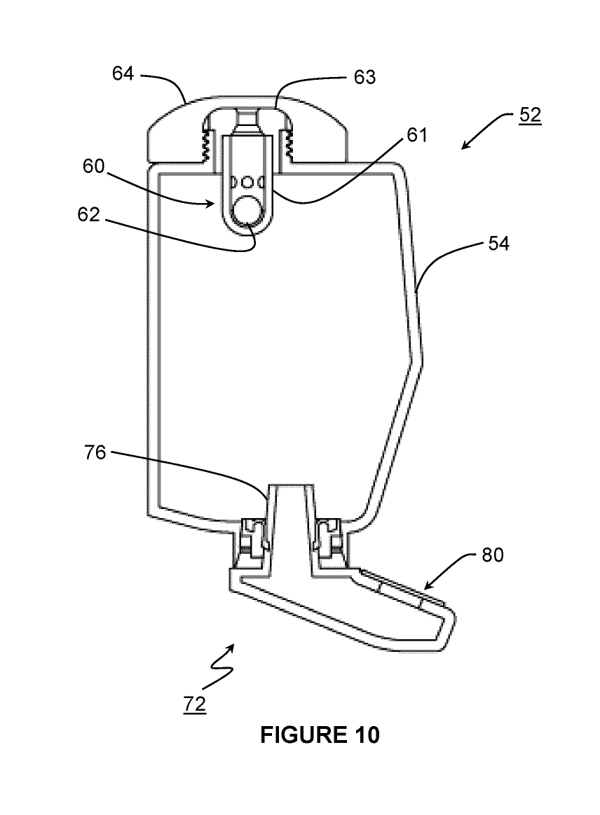

FIG. 9 shows the atomizer reservoir 72 assembled into the atomizer assembly 56 aligned for mating with the fragrance reservoir 52. The chimney 32 of the atomizer assembly 56 channels mist generated by the atomizer disc 84 upwards and outwards.

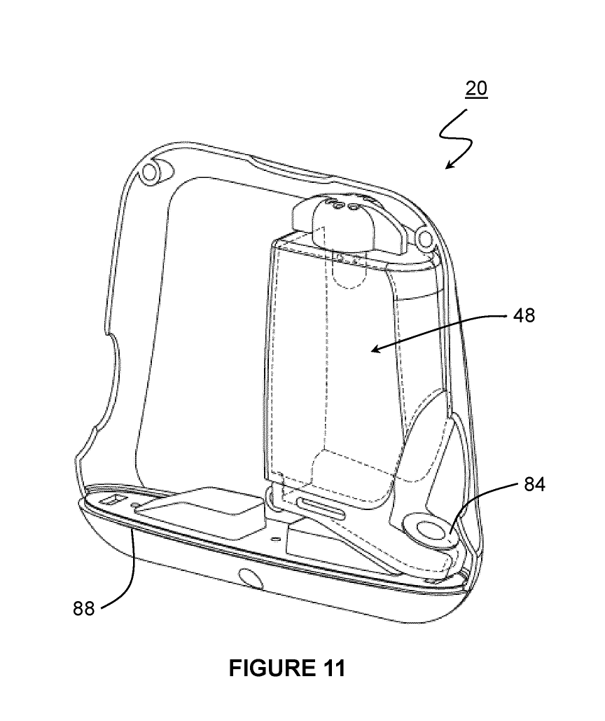

FIG. 10 illustrates the atomizer reservoir 72 after mating its spigot 76 with the petal valve 68 of the fragrance reservoir 52. As shown, the interior of the atomizer reservoir 72 is in fluid communication with the interior of the fragrance reservoir tank 54 to enable the flow of liquid fragrance in the fragrance reservoir tank 54 to the atomizer disc aperture 80, and thereby to the atomizer disc 84 when fitted.

The foregoing configuration is one in which gravity can be used to feed liquid fragrance from the fragrance reservoir tank 54 to the atomizer 84. An alternative approach (not shown) comprises replacing the PET (or other rigid) fragrance reservoir tank 54 with a flexible fragrance reservoir tank. With a flexible fragrance reservoir tank, the ball valve 60 may be omitted as depressurization of the fragrance reservoir tank is prevented due to collapsing of the fragrance reservoir 52 as the atomizer 84 emits liquid fragrance.

The ball valve 60 and the sealing cap 64 are shown in greater detail. The ball valve 60 includes a clear tube extending into the fragrance reservoir tank 54. At least one air hole 61 perforates the tube inside the fragrance reservoir tank 54. The bottom of the tube is sealed to retain a ball 62 moveably disposed therein. The ball 62 has a diameter that is smaller than the inside diameter of the tube to permit travel of the ball along the interior of the tube. A rubber retaining insert 63 holds the clear tube in a neck of the fragrance reservoir tank 54 and has a beveled opening at its top end.

A sealing cap 64 is secured onto a neck of the fragrance reservoir tank 54 via threading around the outer circumference thereof and has perforations through its top surface arranged in a ring surrounding a continuous central surface that aligns with the opening in the rubber retaining insert 63 when the sealing cap 64 is secured on the fragrance reservoir tank 54. The sealing cap 64 can be rotated between a sealing position and an operating position. In the sealing position, used when the fragrance reservoir 52 is not deployed inside the fragrance dispenser 20, the perforations in the sealing cap 64 are pressed against a top surface of the rubber retaining insert 63 around the opening, and the continuous surface between the perforations of the sealing cap 64 is held against the top of the opening of the rubber retaining insert 63 to seal it, thereby preventing evaporation of the liquid fragrance in the fragrance reservoir tank 54 and leakage during transport and storage of the reservoir and in the case of accidental dropping.

In the operating position, used when the fragrance reservoir 52 is deployed inside the fragrance dispenser 20, a gap is opened between the sealing cap 64 and the top of the rubber retaining insert 63, enabling ambient air to flow through the perforations in the sealing cap 64, the gap, the opening of the rubber retaining insert 63, and the air holes 61 into the fragrance reservoir tank 54. As liquid fragrance in the fragrance reservoir tank 54 is consumed, air flows into the fragrance reservoir tank 54 to replace it. This ensures that a vacuum is not created inside the fragrance reservoir tank 54, as this would inhibit the drawing of liquid fragrance from the fragrance reservoir tank 54. When the fragrance reservoir 52 is tilted substantially upside-down, the ball 62 travels via gravity to the top of the tube and forms a seal with the beveled opening of the rubber retaining insert 63 to block the flow of air into and the flow of liquid fragrance out of the fragrance reservoir tank 54. Such a configuration further aids in preventing leaking of the liquid fragrance during transport and storage of the fragrance reservoir 52 and in the case of accidental dropping.

In some scenarios where the fragrance reservoir is separate from the atomizer assembly, other types of mating seals between them can be employed. For example, the fragrance reservoir and the atomizer assembly can have corresponding threaded apertures/projections.

FIG. 11 shows a fragrance cartridge 48 within the fragrance dispenser 20 with the front cover 24 removed. The portion of the atomizer assembly 56 rests on a base portion 88 of the fragrance dispenser 20 to which the back cover 28 is secured.

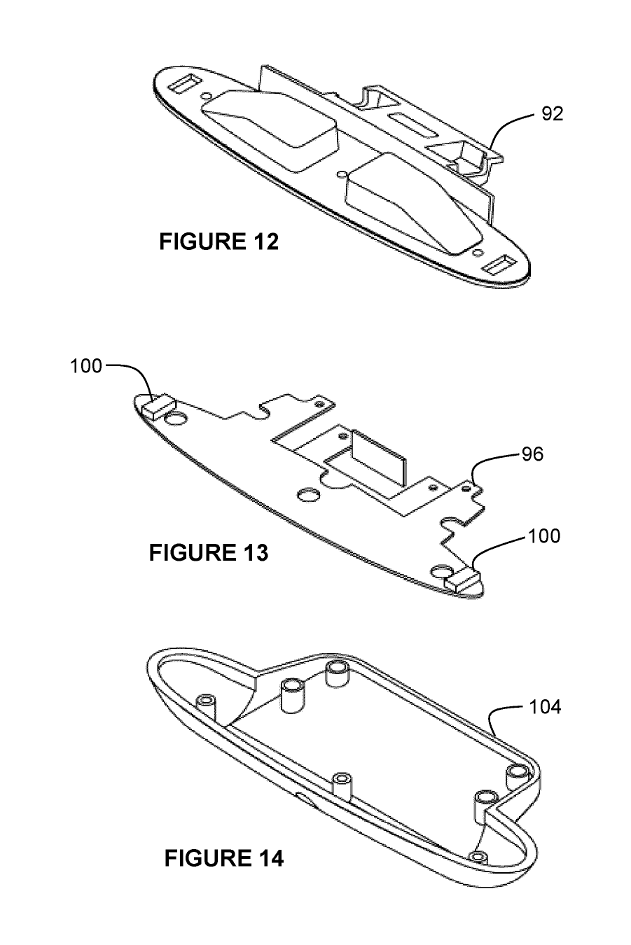

FIG. 12 shows a cartridge deck 92 of the base portion 88 upon which the fragrance cartridges 48 sit within the fragrance dispenser 20.

A circuit board 96 that sits under the cartridge deck 92 and controls operation of the fragrance dispenser 20 is shown in FIG. 13. The circuit board 96 includes a processor, power management circuitry coupled to a pair of contact blocks 100, a wireless radio for wireless communication, and storage for registering the configuration of the fragrance dispenser 20. The wireless radio can be for communication via a number of suitable standards, such as Bluetooth, Wi-Fi, etc. An atomizer driving circuit for powering the atomizer disc 84 can form part of the circuit board 96 or can be a separate printed circuit board.

FIG. 14 shows a bottom cover 104 that is secured to the back cover 28. The cartridge deck 92 and the circuit board 96 are secured to the bottom cover 104 via a set of screws.

As will be understood, when assembled, the contact blocks 100 of the circuit board 96 protrude through openings shown in the cartridge deck 92.

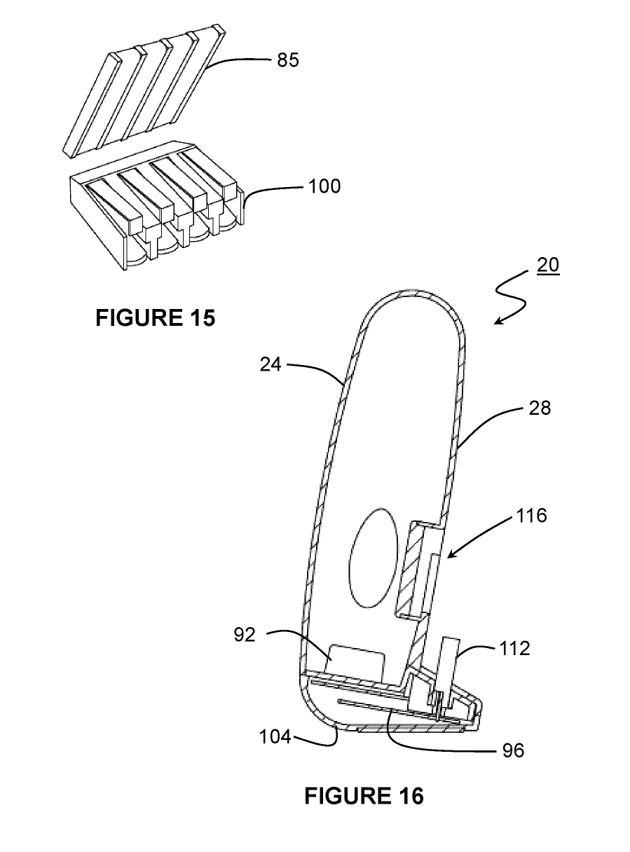

FIG. 15 shows the contact plate 85 that is affixed to the bottom of the atomizer assembly 56 and electrically coupled to the atomizer disc 84. When the atomizer assembly 56 is positioned inside of the fragrance dispenser 20 atop the cartridge deck 92, the contact plate 85 comes into electrical contact with the contact block 100 of the circuit board 96 to power the atomizer disc 84.

FIG. 16 is a side sectional view of the fragrance dispenser 20 without fragrance cartridges 48 housed therein. The cartridge deck 92, the circuit board 96, and the base cover 104 are shown in their assembled state with the front cover 24 and the back cover 28. A male universal serial bus (“USB”) connector 112 is shown coupled to the circuit board 96 and projecting up out of the rear of the base cover 104. The fragrance dispenser 20 is powered via the male USB connector 112. A vertical keyhole slot 116 is provided for aligning and securing the power adapter 36 to the back cover 28.

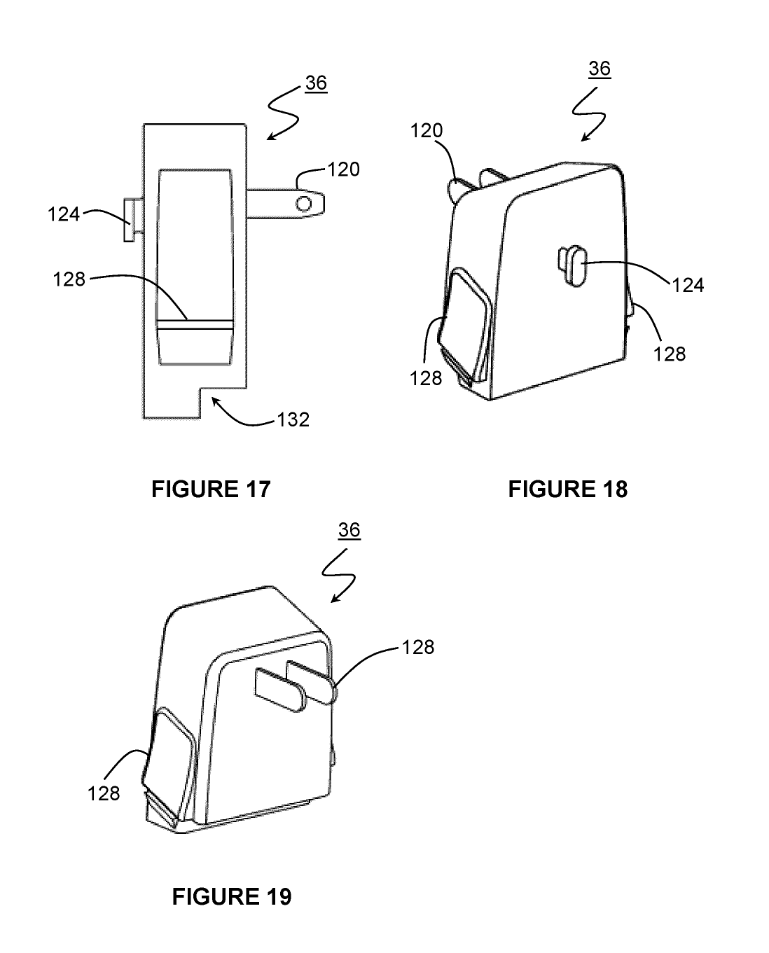

FIGS. 17, 18, and 19 show the power adapter 36 having a pair of electrical pins 120 for drawing current from a standard electrical outlet. A keyhole pin 124 protrudes from the front of the power adapter 36. Two spring-loaded clips 128 are disposed on either side of the power adapter 36, and a female USB connector 132 is positioned on a bottom side thereof.

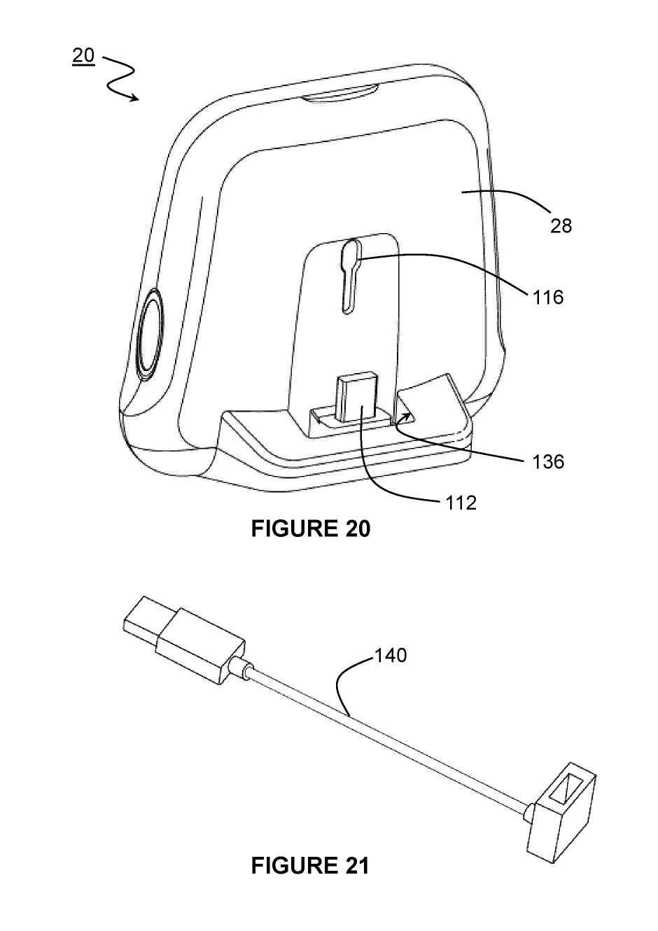

FIG. 20 shows the rear of the fragrance dispenser 20 having the vertical keyhole slot 116 and the male USB connector 112. Two ridges 136 are provided on either side of a recess adjacent the male USB connector 112.

The power adapter 36 can be coupled to the circuitry of the fragrance dispenser 20 in two different ways.

In a first configuration, the power adapter 36 can be secured to the back cover 28 so that the fragrance dispenser 20 can be directly plugged into an electrical outlet. In order to do this, the keyhole pin 124 of the power adapter 36 is aligned with and inserted into the vertical keyhole slot 116 in the back cover 28. The female USB connector 132 of the power adapter 36 can then engage and mate with the male USB connector 112 of the base portion 88 of the fragrance dispenser 20. When the female USB connector 132 and the male USB connector 112 are fully mated, the spring-loaded clips 128 of the power adapter 36 engage the clip holes 136 in the recess of the base portion 88 of the fragrance dispenser 20 to secure the power adapter 36 to the base portion 88 and the back cover 28.

In a second configuration, the power adapter 36 is electrically connected to the fragrance dispenser 20 via a USB cable such as USB cable 140 shown in FIG. 21. As will be understood, the length of the cable can be varied. In this configuration, the fragrance dispenser 20 can be positioned away from an electrical outlet such as on the floor on a table.

By using standard male and female USB connectors on the power adapter 36 and the base portion 88 of the fragrance dispenser 20, together with the keyhole pin and slot, and the spring-loaded clips 128 and clip holes 136, the power adapter 36 can be removed from and connected to the main body of the fragrance dispenser 20 via the standard USB cable 140.

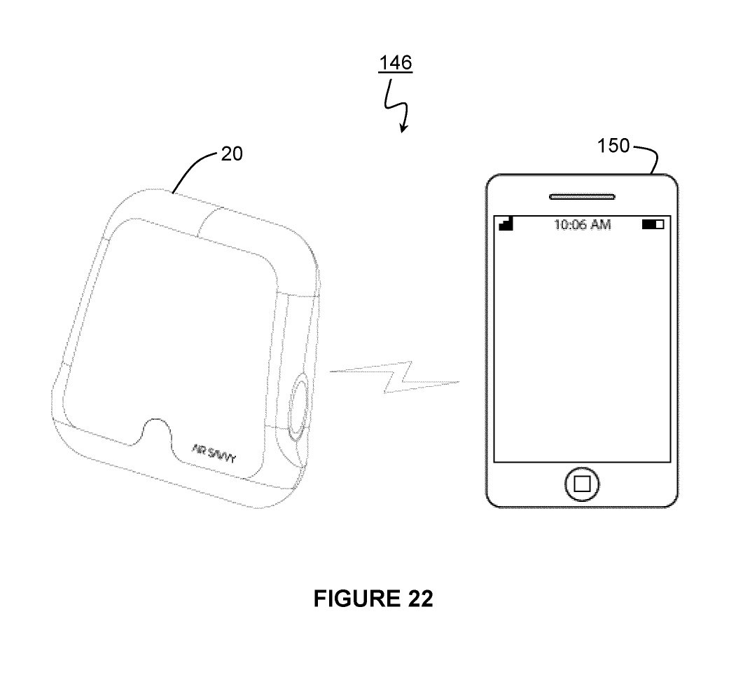

A fragrance dispenser system 146 is shown in FIG. 22, and includes the fragrance dispenser 20 and a mobile device. In the illustrated embodiment, the mobile device is a smartphone 150. The fragrance dispenser 20 includes wireless network radio to permit remote control of the fragrance dispenser 20 via the smartphone 150. Through an application that is installed on the smartphone, a user may remotely control the operation of the fragrance dispenser 20, including the intensity for each of the two fragrances in the fragrance cartridges 48, a schedule for varied operation of the fragrance dispenser 20, etc.

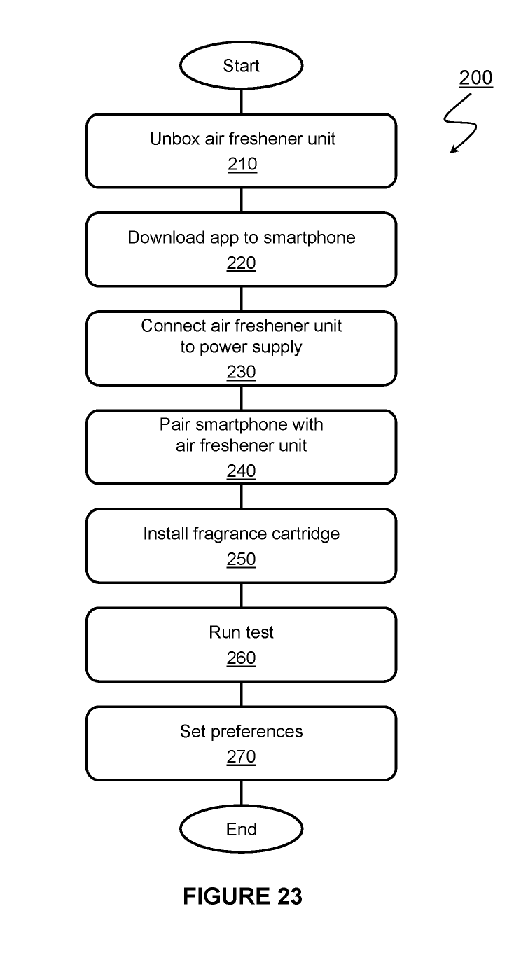

FIG. 23 shows a method 200 of initializing the fragrance dispenser system 146 shown in FIG. 22. The method 200 commences with the unboxing of the fragrance dispenser 20 (210). A remote control application for the fragrance dispenser 20 is then downloaded and installed on the smartphone 150 (220). The application executing on the smartphone 150 guides a user through the remainder of the initialization process step by step. The fragrance dispenser 20 is then connected to a power supply (230). As noted above, the power adapter 36 is either coupled directly to the male USB connector 112 of the fragrance dispenser 20 or is coupled to it via the USB cable 140 of FIG. 21. The power adapter 36 is then plugged into a standard electrical outlet. Once the fragrance dispenser 20 is powered, the smartphone 150 can be paired with the fragrance dispenser 20 (240). In order to pair the smartphone 150 with the fragrance dispenser 20, the smartphone 150 is temporarily connected to the fragrance dispenser 20 via Wi-Fi or Bluetooth by the user. Upon opening the application on the smartphone 150, it reads information from the fragrance dispenser 20 and registers it in its list of fragrance dispensers 20 that it manages. The user is then prompted for information about a local Wi-Fi network. Upon entering the information, the information is communicated to the fragrance dispenser 20 and it connects to the specified Wi-Fi network. The application on the smartphone 150 then directs the user to revert back to the local Wi-Fi network over which it can then communicate with the fragrance dispenser 20. Once the pairing is completed, a fragrance reservoir 52 is installed in the fragrance dispenser 20 (250). The fragrance is identified via a code presented on the packaging of the fragrance reservoir 52 and is manually inputted into the application executed by the smartphone 150 by the user, or through a QR type code on the packaging captured through a photograph taken within the application using a camera of the smartphone 150, or by an RFID tag on the fragrance reservoir 52 and a corresponding RFID reader on the circuit board 96. This enables registration of the liquid fragrances that are being loaded into the fragrance dispenser 20. Alternatively, the user may be prompted by the application to select which liquid fragrance has been loaded into the fragrance dispenser 20. The fragrance dispenser 20 is packaged with two atomizer assemblies 56, and may be bundled with one or two fragrance reservoirs 52 that are filled. If none are provided, fragrance reservoirs 52 may be purchased separately. In order to use the fragrance reservoirs 52, the sealing cap 64 is rotated counter-clockwise to allow airflow through the ball valve 60. The fragrance reservoir 52 is then loaded atop of an atomizer assembly 56 inside of the fragrance dispenser 20 and the front cover 24 is closed.

A test is then run (260). The user selects to run a test via the application on the smartphone 150. The user taps two buttons presented on the user interface of the application corresponding to the liquid fragrances loaded in the left and right sides of the fragrance dispenser 20. A tap of the button releases a short burst of fragrance from the respective side, demonstrating that the fragrance is being successfully atomized. Once the test is run, the user can set preferences for the fragrance dispenser 20 via the smartphone 150 (270). Such preferences can include a room identifier for the fragrance dispenser 20 to help identify the fragrance dispenser 20 (as multiple fragrance dispensers can be controlled via the same smartphone 150), an operating schedule for the fragrance dispenser 20 by day of the week and by time of day, settings for the intensity of the fragrances from each of the two fragrance cartridges 48, etc.

Upon setting preferences for the fragrance dispenser 20, initialization of the fragrance dispenser system 146 is complete.

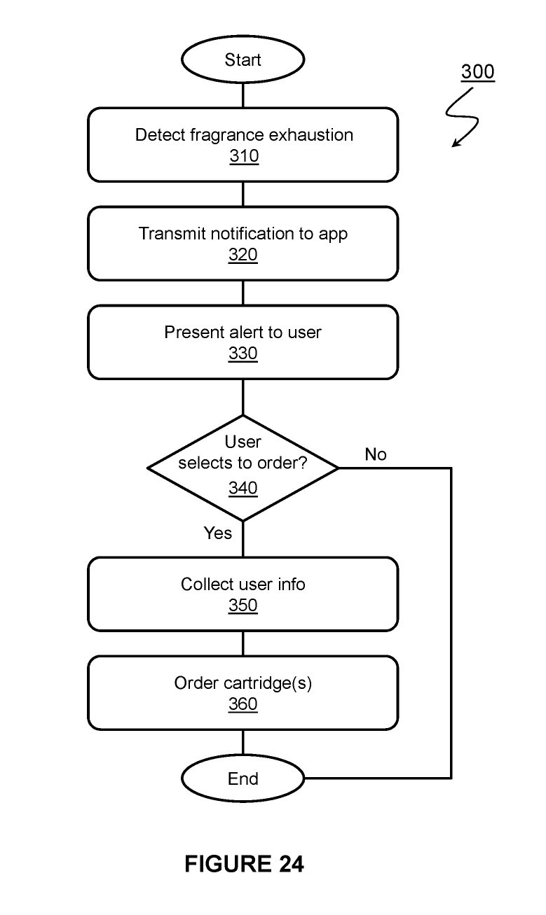

FIG. 24 shows the general method of ordering a replacement fragrance reservoir 52 using the fragrance dispenser system 146 of FIG. 22. The method 300 commences with the fragrance dispenser 20 detecting that the liquid fragrance in the fragrance cartridge 48 is exhausted (310). A filled fragrance reservoir 52 is assumed to contain enough liquid fragrance for a pre-determined activation time of the atomizer 84. Each time a burst of liquid fragrance is emitted, the time during which the atomizer 84 was activated for is tallied in a memory of the circuit board 96 for the particular fragrance reservoir 52. When the processor on the circuit board 96 determines that a threshold value has been reached for a particular fragrance reservoir 52, it deems the fragrance reservoir 52 to be sufficiently low in liquid fragrance such that its replacement/replenishment should be planned to enable continued use.

The fragrance dispenser 20 then transmits a notification to the application executing on the smartphone 150 (320). The application on the smartphone 150, when executed, polls the fragrance dispenser 20 over the wireless network to determine its status information. This status information includes its current configuration, and whether the liquid fragrance in one of the fragrance cartridges 48 is running low and needs replenishing. If the received status information indicates that a fragrance cartridge 48 is should be replaced, the application executing on the smartphone 150 presents an alert screen to the user (330). The alert screen presents a button asking the user if they would like to order a refill fragrance cartridge 48 (340). Upon activation of the button for the first time by a user, the application presents another screen wherein the user can make a liquid fragrance and quantity selection, and will then be prompted for shipping and billing information. (350). Upon completing the second screen and activating an order button after confirming the order, the order is processed (360). For subsequent refills, the user may choose to use the information previously provided or may edit the information,

Once a replacement fragrance reservoir 52 is obtained, the front cover 24 is removed from the fragrance dispenser 20 and the empty fragrance reservoir 52 is withdrawn from the interior of the fragrance dispenser 20 and discarded, recycled, or, where possible, refilled. The fragrance dispenser 20 includes a micro sensor for determining when the front cover 24 is removed and terminates operation of the atomizer disc 84. The sealing cap 64 at the top of the replacement fragrance reservoir 52 is then turned to enable airflow into the fragrance reservoir 52 via the ball valve 60.

Once the sealing cap 64 is opened, the petal valve 68 of the replacement fragrance reservoir 52 is then aligned with and pushed onto the spigot 76 of the atomizer assembly 56. The front cover 24 is then replaced, after which the fragrance dispenser 20 is then ready for continued operation.

By making the fragrance reservoir 52 separable from the atomizer assembly 56, the cost of replacement fragrance reservoirs can be reduced as an atomizer assembly need not be included.

The fragrance reservoirs can include an identifier of the liquid fragrance contained therein. The identifier may be a code on an RFID chip, a memory coupled to an interface, a label, etc. The fragrance dispenser can then automatically determine the fragrance types loaded.

The fragrance dispenser 20 may also or alternatively indicate that one or more of the fragrance reservoirs is running low on liquid fragrance by means of one or more indicator lights, lighting patterns or colors, sounds, vibration, etc. It may also be configured to push a message via email, SMS, etc. by connecting to a local server or a server on the Internet via the local network to which it is connected.

The fragrance dispenser may alternatively communicate with the smartphone and/or one or more servers on the Internet via Bluetooth or any other suitable wireless or wired communications method. For example, the fragrance dispenser may be connected to a local router via an Ethernet cable.

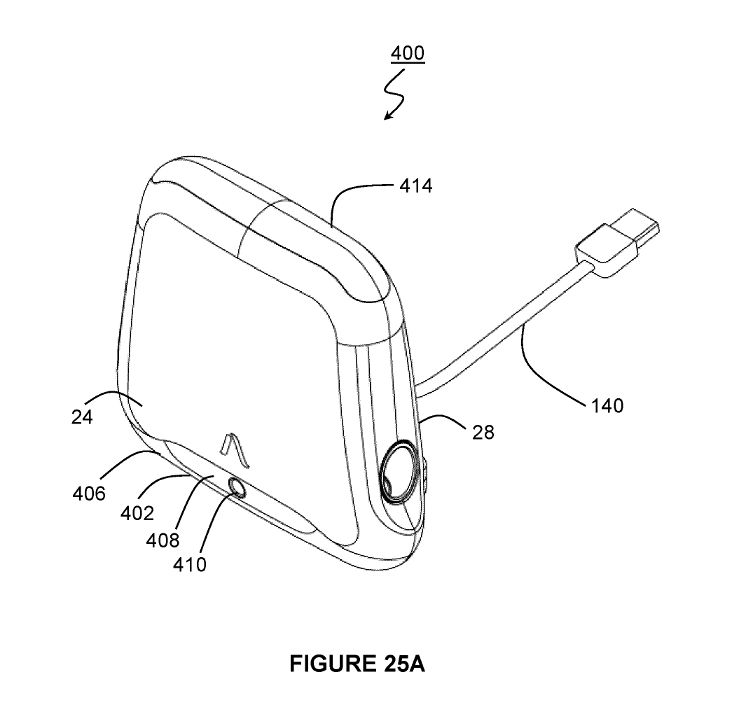

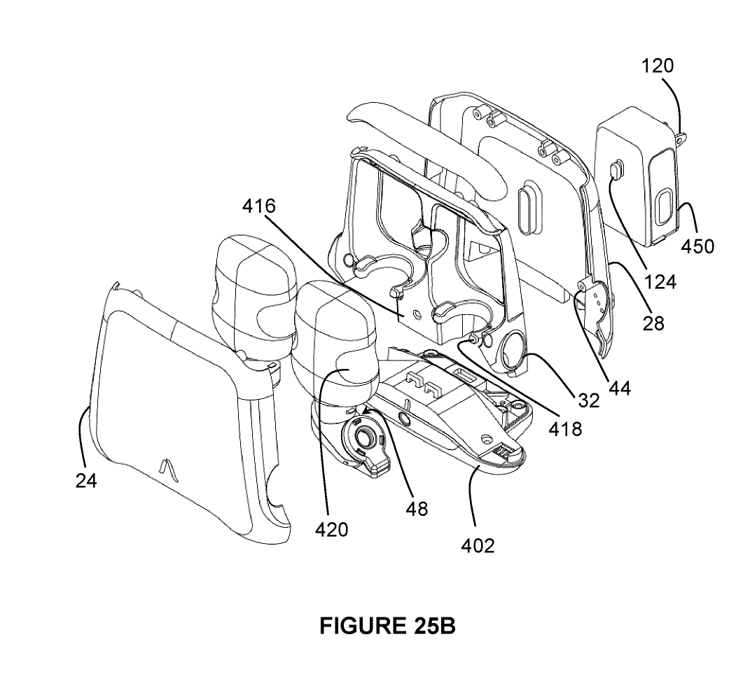

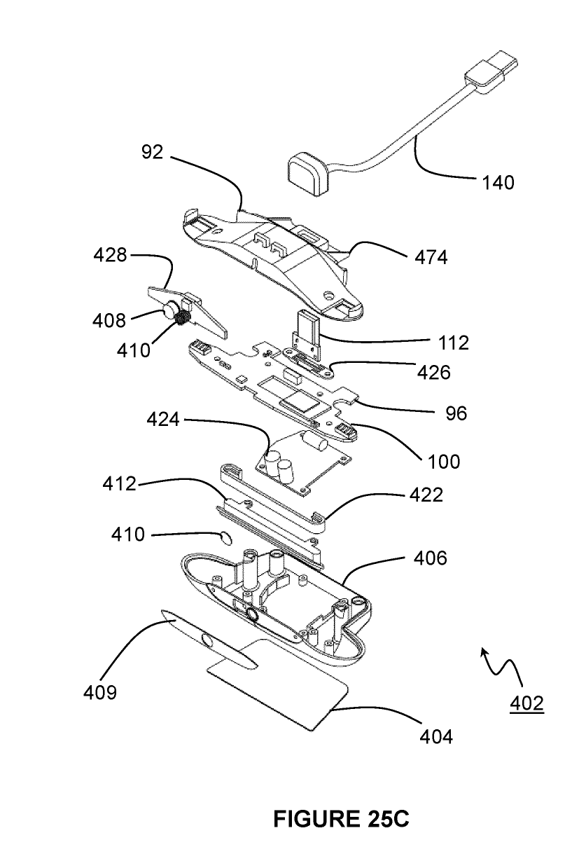

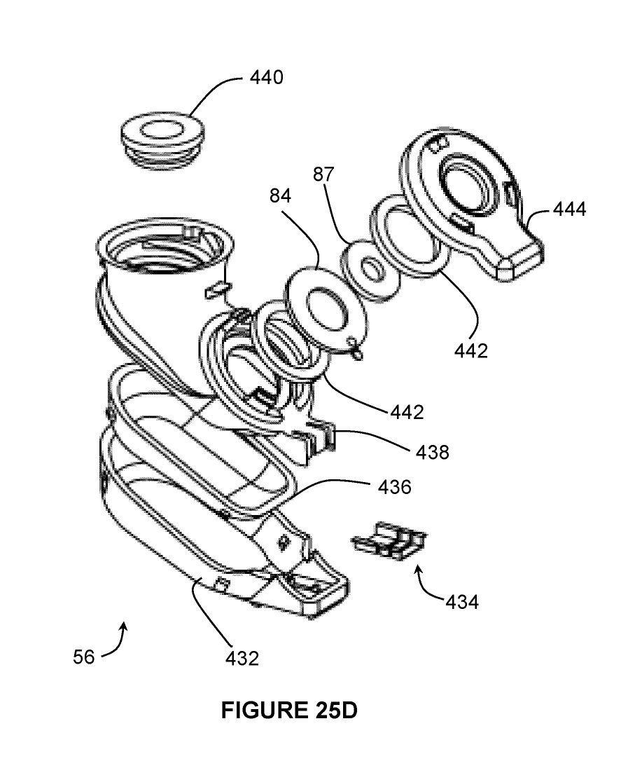

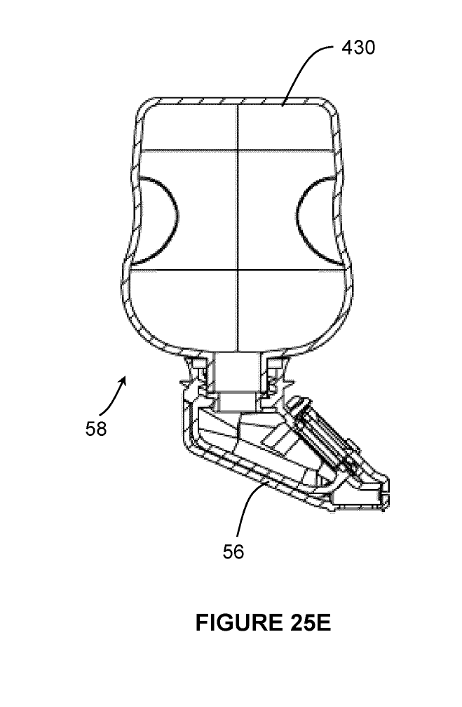

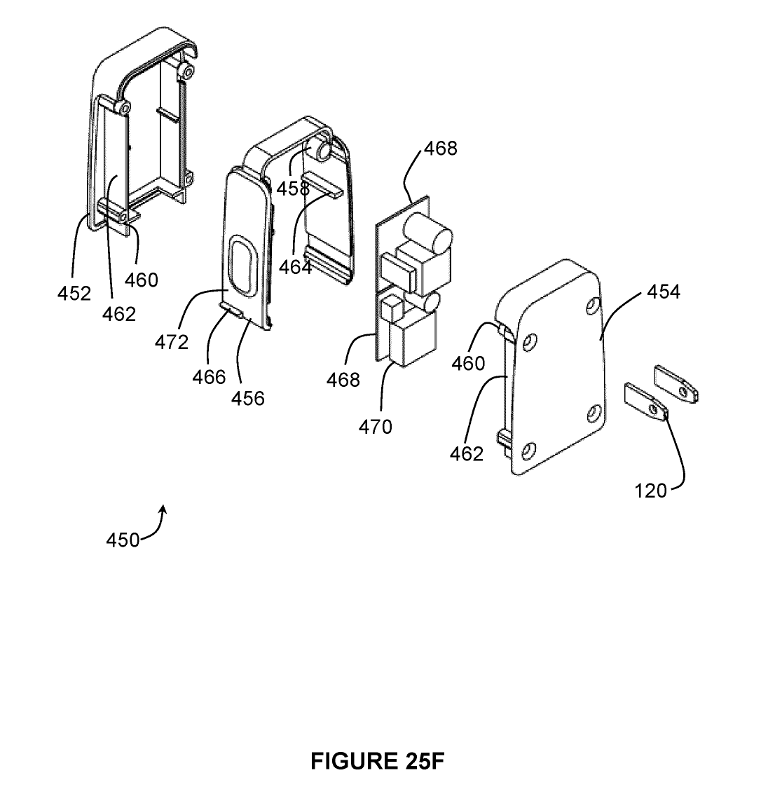

FIGS. 25A to 25F show an alternative dispenser 400 for dispensing fragrances. The alternative dispenser 400 can have one or more of the elements of the fragrance dispenser 20 and any other variations of fragrance dispensers described herein. Similarly, one or more of the elements of the alternative fragrance dispenser 400 can be used with the fragrance dispenser 20 and any other variations of fragrance dispensers described herein. Items shown with a reference number in FIGS. 25A to 25F that also appears in another Figure indicate a similar component.

The base 402 of the alternative dispenser 400 includes a motion sensor 408 attached to a circuit board 96 (see FIG. 25C) inside of the fragrance dispenser 400. The motion sensor 408 may be a passive infrared (PIR) based motion detector, sometimes call a passive infrared detector (PID). The PIR-based motion detector has a sensor body containing a PIR sensor. The PIR sensor measures infrared radiation (heat energy) entering the sensor from objects in the sensor field of view. The PIR sensor may be a thin film of material that generates a voltage when it receives infrared radiation. A person passing entering the field of view of the PIR-based motion detector causes an increase in the voltage produced by the sensor. Sensor voltage or variations in the sensor voltage can be interpreted, for example through an algorithm or circuitry, to indicate that a person has moved into, is present within, or has left the sensor field of view. Optics, for example a Fresnel lens, can be used to alter, for example widen, the field of view. A filter over the sensor may be used to limit incoming radiation to wavelengths characteristic of humans to avoid detecting other heat energy sources such as pets or operating appliances.

In the examples illustrated, the motion sensor is able to detect a person at a maximum distance of 10-15 feet within an arc of between 90 and 180 degrees. This field of view is generally consistent with sensing a person in the same room as the alternative dispenser 400. For voltage signals produced when a person is within that field of view, software or circuitry in the alternative fragrance dispenser 400 or a computer communicating with the alternative fragrance dispenser is optionally configured to distinguish between a lower voltage range and a higher voltage range. The lower voltage range corresponds with a person in the field of view but not close to the motion sensor 408. The higher voltage range corresponds with a person close to the motion sensor 408, for example within 1.5 m of the voltage sensor 408. The alternative dispenser can be configured to emit one or more bursts of fragrance while a person is in the field of view, for example one burst every 5 to 20 minutes. Alternatively or additionally, the alternative dispenser 400 can be configured to delay or cancel a burst while a person is close to the motion sensor 408. Alternatively or additionally, the alternative dispenser 400 can be configured to delay or cancel a burst if any other instruction would lead to an excessive number of bursts within a period of time, for example because a person leaves and re-enters the field of view over and over within a short period of time.

In another alternative, the motion sensor 408 can be used to detect motion in front of the fragrance dispenser 400 and only operate the atomizer disc when motion is detected and for a set period of time thereafter. In this manner, liquid fragrance can be conserved. Alternatively, the fragrance dispenser 400 can be configured to not operate for a set period of time when motion, or a certain type of motion, is detected to avoid, for example, releasing bursts of fragrance in the presence of pets.

The operation of the fragrance dispenser 400 in response to data from the motion sensor 408 can be configured or controlled via an application executing on a smartphone or a remote computer as generally described further below. For further example, the period of time during which the fragrance dispenser 400 is operated after motion detection can be set via the application. Further, the fragrance dispenser 400 can be configured via the application to switch atomizing liquid fragrance from a first fragrance cartridge to atomizing liquid fragrance from a second fragrance cartridge for a set period of time after motion is detected.

Optionally, information from the motion sensor 408 can be conveyed and stored in a smartphone or remote computer. This enables a machine-learning mode wherein the smartphone or remote computer learns the user's activation pattern and seeks to replicate it. For example, a person may have an alternative dispenser 400 in their kitchen, and regularly press a burst button 410 to emit one burst of fragrance when entering the kitchen in the morning but at no other time of the day. The computer may learn this pattern and emit one burst when the motion sensor 408 first indicates that a person is in the field of view if before noon, but at no other times. In another example, a person may have an alternative dispenser 400 in their kitchen and another alternative dispense 400 in their bedroom. This person frequently passes through the kitchen and then pushes the burst button 410 to emit one burst in the bedroom in the evening. The smartphone or remote computer, connected directly or indirectly to both alternative dispensers 400, may learn this pattern and proactively emit one burst in the bedroom if after 8 pm and the person has just passed through the kitchen.

The burst button 410 as mentioned above enables a user to command the alternative dispenser 400 to emit a burst of fragrance from a fragrance cartridge 48. As discussed further below, the alternative dispenser 400 can be configured to release a single burst or a series of burst from the left or the right fragrance cartridge 49 when the burst button 410 is touched. The burst button 410 is connected to the circuit board 96 within the alternative dispenser 400. The burst button 410 shown is a touch capacitive disc but any other type of button capable of giving a signal when touched to an electronic circuit may be used.

Optionally, one or more portions of the alternative dispenser 400 can be outfitted with illumination. The illumination can be of variable color and its schedule of operation and characteristics, including color and pattern of light, can be predetermined or configured via an application executing on a smartphone or remote computer. The operation schedule of the illumination may alternatively be coordinated with the operation schedule of the release of fragrances. In the example shown, a light strip 412 (see FIG. 25C) is provided in the base 402 of the alternative dispenser 400 and shines though part of a bottom cover 406 of the base 402.

A communications antenna wire of the circuit board 96 optionally extends under a plastic cap 414 on the outside of the alternative dispenser 400, for example on the top of the alternative dispenser 400. The communications antenna may be, for example, a WiFi antenna or Bluetooth antenna or both. Alternatively, a communications antenna may extend across a surface of the circuit board 96 or dangle from the circuit board 96. However, in the example shown, the front cover 24 and back cover 28 are made of aluminum, which would dampen the RF range of an antenna located between the covers 24, 28. Extending the antenna wire to the bottom of cap 414 improves radio reception and range.

FIG. 25B shows parts of the alternative dispenser 400. The structure is similar to the dispenser 20 but includes a cartridge cradle 416. The cartridge cradle 416 provides chimneys 32 for two fragrance cartridges 48 in one unit. The cartridge cradle 416 can rest on the base 402 or be suspended from bosses 44 of the back cover 28. The fragrance cartridges 48 in turn rest on the cartridge cradle 416. Integrated spring fingers 418 of the cartridge cradle 416 flex when a fragrance cartridge 48 is inserted into the cartridge cradle 416 and wrap around the neck of the fragrance cartridge 48. This helps to hold the fragrance cartridge 48 in place while the alternative dispenser 400 is in used. However, a person can remove the front cover 24 and then pull a used fragrance cartridge 48 out by overcoming the grasp of the spring fingers 418. Detents 420 in the fragrance cartridge 48 help a user grip the fragrance cartridge 48 while installing or removing it from the cartridge cradle 416.

FIG. 25C shows parts of the base 402 of the alternative dispenser 400. The bottom cover 406 is the primary component visible from outside of the alternative dispenser 400. An optional rubber sheet 404 can be adhered into a recess in the bottom cover 406 to help prevent the bottom cover 406 from sliding on smooth surfaces. Other parts of the alternative dispenser 400 include motion sensor 408, lens 409, burst button 410, light strip 412, LED shield 422, driving circuit 424, circuit board 96, cartridge deck 92, USB plate 426, male USB connector 112 and front panel 428. Burst button 410 as shown is a spring with a touch capacitive disc that can be contacted through a hole in the bottom cover 406 although other forms of buttons or switches can be used. Motion sensor 408 is a PIR-based motion sensor that takes in IR radiation through a hole in bottom cover 406 that is covered by the lens 409. Lens 409 may be a Fresnel lens. Light strip 412 shines light through an opening, not visible in FIG. 25C, near the bottom of the bottom cover 406. Circuit board 96 includes electronic circuits including flash memory, a Bluetooth radio and a WiFi radio. Driving circuit 424 may be considered part of the circuit board 96 and includes the higher current components used to drive the piezoelectric atomizers in the fragrance cartridges 48.

FIG. 25D shows parts of an atomizer assembly 56 used with the alternative dispenser 400. The atomizer assembly 56 includes a contacts holder 432 and electrical contacts 434. An atomizer chamber bottom 436 and atomizer chamber top 438 form an atomizer reservoir when adhered together. A cartridge body O-ring 440 provides a seal between the atomizer assembly 56 and an alternative reservoir 430 (shown in FIG. 25E). The atomizer disc 84 is held between two atomizer O-rings 442 which are compressed between the atomizer chamber top 438 and the atomizer cap 444. An optional wicking pad 87 is placed on top of the atomizer disc 84 but outside of its active area. The wicking pad 87 is made of absorbent material.

FIG. 25E shows an alternative fragrance reservoir 430 connected (for example by half turn screw threads as shown) to an atomizer assembly 56 to form a fragrance cartridge 58. Make this connection, a user holds the alternative reservoir 430 in an inverted position relative to what is shown in FIG. 25E and removes a threaded cap and foil seal from the alternative reservoir 430. The cap and seal are provided to prevent leaks when shipping or storing the alternative reservoir 430. The user places the atomizer assembly 56 over the alternative reservoir 430. The user then twists the atomizer assembly 56 or the alternative reservoir 430, for example by 180 degrees, to attach and seal the atomizer assembly 56 to the alternative reservoir 430. The user then flips the fragrance cartridge 48 over so that the atomizer assembly 56 is below the alternative reservoir 430. The user can then insert the fragrance cartridge 58 into the alternative dispenser 400.

The alternative reservoir 430 differs from the previously described fragrance reservoir 52 in that it has no vent. The fragrance cartridge 58 provides a sealed enclosure except for the pores in the atomizer disc 84. The free surface of the fragrance in the fragrance cartridge 58 remains above the top of the atomizer disc 84 during operation. A small volume of fragrance below the top of the atomizer disc 84 is not usable. Gravity delivers fragrance to the atomizer disc 84. The partial vacuum does not become overly large. Bubbles are sometimes observed forming on the back of the atomizer disc 84 while fragrance is being emitted. Without intending to be limited by theory, it is possible that some air enters the fragrance cartridge 58 through the atomizer disc 84 while power is supplied to the atomizer disc 84. However, it appears that a partial vacuum forms or is maintained at least between when bursts of fragrance are emitted since no fragrance, or at least less fragrance relative to the fragrance reservoir 52, is observed in the wicking pad 87 with the alternative reservoir 430 between when bursts of fragrance are emitted. The wicking pad 87 is made of absorbent material and collects any liquid fragrance that appears on the front of the atomizer disc 84 between bursts. With the alternative reservoir 430, some fragrance is observed in the wicking pad 87 when the fragrance cartridge 58 is first inverted. The absorbed fragrance later evaporates and the wicking pad 87 tends to dry out and then remain dry in use.

FIG. 25F shows an exploded view of a transformer 450. The transformer 450 is an alternative to power adapter 36 and has the same components and operates in the same way except as described below. The body of the transformer 450 is made up of a transformer front 452, a transformer back 454 and a transformer center 456. The transformer front 452 has a keyhole pin 124 (see FIG. 25B) to engage with a keyhole slot 116 on the alternative dispenser 400 as described for the dispenser 20. The transformer back 454 has electrical pins 120 to engage with an electrical outlet. The transformer back 454 is attached to the transformer front 452 with the transformer center 456 in between using screws (not shown) that pass through bosses 460 in the transformer back 454 and thread into bosses 460 in the transformer front 452. A circuit board 468 is also provided between the transformer front 452 and the transformer back 454. Parts of the bosses 460 pass through tubes 458 in the transformer center 456. Ribs 464 of the transformer center 456 bear against side panels 462 of the transformer front 452 and transformer back 454.

The sides 472 of the transformer center 456 are flexible and bend around the ribs 464 when pressed inwards by a user's fingers. Latches 466 at the bottom of the sides 472 snap into engagement with slots 474 in the cartridge deck 92 (see FIG. 25C) of the base 402 of the alternative dispenser 400 when the keyhole pin 124 of the transformer 450 is slid downwards in the keyhole slot 116. This attaches the transformer 450 to the base of the alternative dispenser 400 and causes a female USB connector 470 on the circuit board 96 to engage with male USB connector 112 of the dispenser base 402. The attachment is strong enough to allow the alternative dispenser 400 to be mounted on a wall, suspended from the transformer 450 when it is plugged into an electrical outlet. Conversely, pressing the sides 472 inwards releases the latches 466 so that the transformer 450 can be pulled upwards. The keyhole pin 124 can then be removed from keyhole slot 116 to release the transformer 450 from the base of the alternative dispenser 400. The alternative dispenser 400 can then be placed on a table or other surface and connected to the transformer 450 through USB cable 140. Optionally, the USB cable 140 can be used to power the alternative dispenser 400 from, for example a laptop computer or generic charger. The transformer 450 produces slightly more than normal USB voltage to power light strip 412. When powered at normal USB voltage, light strip 412 is disabled.

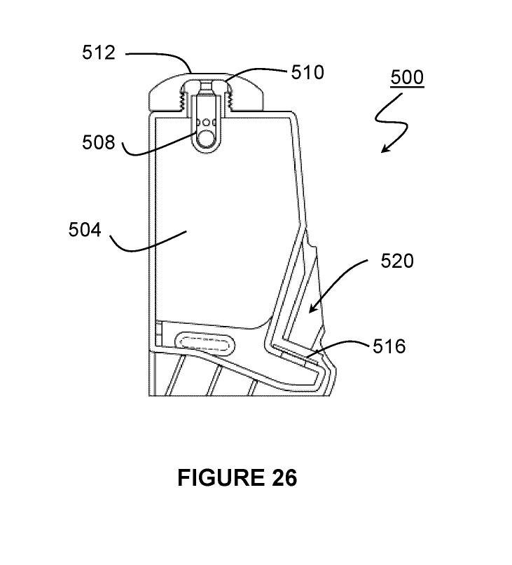

FIG. 26 shows a sectional view of a fragrance cartridge 500 in accordance with an alternative embodiment. In this embodiment, the fragrance cartridge 500 includes a fragrance reservoir tank 504 that is integral with the atomizer assembly. This construction may be desirable in some scenarios to reduce the probability of liquid fragrance spillage. A ball valve 508 similar to that employed in the fragrance reservoir 52 of FIG. 5 is situated at the top end of the fragrance reservoir tank 504, and is held inside a neck of the fragrance reservoir tank 504 via a rubber retaining insert 510 thereof. The rubber retaining insert 510 has an aperture through its top surface aligned with an opening at the top of a tube of the ball valve 508. A sealing cap 512 having perforations in its top surface is threadedly secured atop of the neck of the fragrance reservoir tank 504. The sealing cap 512 is operated like the sealing cap 64 of FIG. 10 to prevent and permit airflow through the ball valve 508 and into the fragrance reservoir tank 504. An atomizer disc 516 is positioned on an upper surface of a lower end of the fragrance reservoir tank 504 to mist liquid fragrance contained therein. A chimney 520 directs misted liquid fragrance into the ambient environment.

Other mechanisms for permitting air to enter into the fragrance reservoir can be employed in place of the ball valve in the fragrance cartridge 48 or the fragrance cartridge 500. For example, a Gore-Tex™ membrane can span over an aperture on the top of the fragrance reservoir to permit ambient air to enter the fragrance reservoir, while preventing liquid fragrance from inadvertently spilling out should the fragrance dispenser be knocked over.

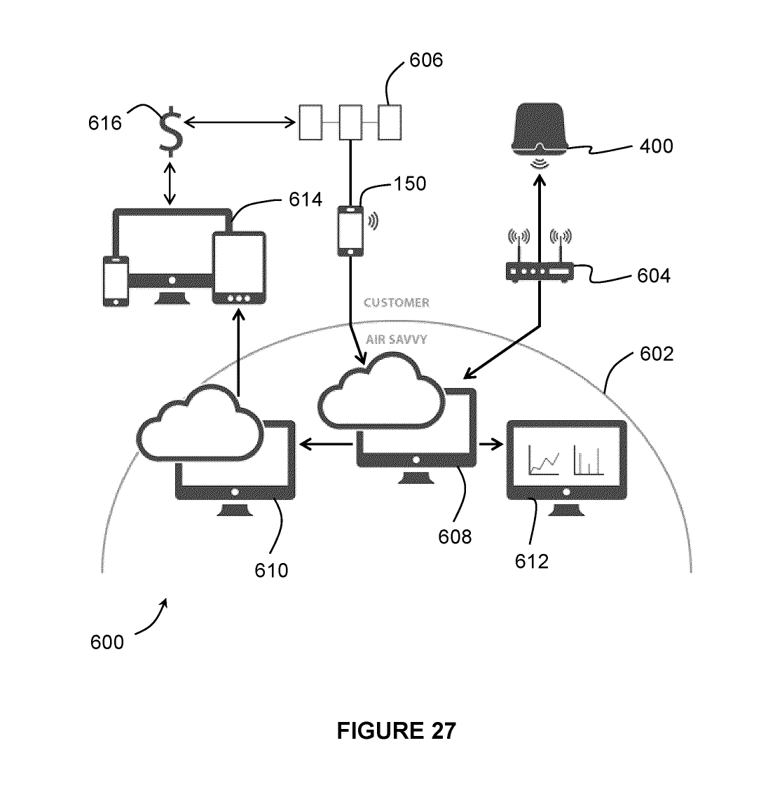

FIG. 27 illustrates the architecture of the alternative system 600. The alternative system 600 includes one or more alternative dispensers 400. Optionally, the alternative system 600 could include fragrance dispenser 20 or another dispenser. The alternative system 600 also includes a user's computer represented in the example shown by a smartphone 150. The alternative system 600 also includes back end 602 having one or more remote computers, for example servers.

The alternative dispenser 400 is controlled at least in part via the smartphone 150 either directly or through the back end 602. In some examples, the alternative dispenser 400 is controlled at least in part by the smartphone 150 communicating through the back end 602. Communications with the alternative dispenser 400 can be through a router 604, such as a wireless WiFi router, located in a building where the alternative dispenser 400 is located. The router 604 communicates with the alternative dispenser 400, and optionally with the smartphone 150, and is further connected through the Internet to one or more computers of the back end 602. The smartphone 150 can communicate with the back end 602 though the router 604 or through any other Internet linkage available to it, for example a cellular telephone network or a WiFi router in another building.

The smartphone 150 operates an application program (“app”) 606 that displays information related to operation of the alternative dispenser 400 on a screen of the smartphone 150 and accepts commands from the user through the smartphone 150 and optionally stores data related to operation of the alternative dispenser 400, which may include user preferences. Alternatively or additionally, data related to operation of the alternative dispenser 400 may be stored in the back end 602.

The back end 602 can include, on one or more computers (i.e. a servers), a device backend 608, a website back end 610 and an administrative back end 612. The device backend 608 processes instructions between the smartphone 150 and alternative dispenser 400 and may optionally store customer information related to the operation of the alternative dispenser 400. The device backend 608 may also receive, process or store information from the alternative dispenser 400. For example, the device backend 608 may track the amount the number of bursts of fragrance that have been made from a fragrance cartridge so as to provide an estimate of the fill level of the cartridge to the user via the smartphone 150. The website back end 610 supports a website 614 that enables communication with the user. For example, the user can download the app 606 from the back end 602 through the website 614. The user may also order replacement fragrance cartridges 48 through the website 614. Optionally, some communications between the user and the website 616 may occur through an E-commerce computer 616 that, for example, may provide for encrypted or otherwise secure transmission and storage of credit card and other personal information of the user. The administrative back end 612 processes information relevant to the operation of the alternative system 600, for example aggregated information on the purchase or consumption of reservoirs 52, 430 by fragrance type, season or distribution channel that is used to plan manufacture, shipping or warehousing of replacement reservoirs 52, 430.

After being unboxed, the alternative dispenser 400 is plugged in. Once plugged in, the alternative dispenser 400 will start up, initialize its Bluetooth radio, WiFi radio and flash memory, and then idle. LEDs in the light strip 412 in the base 402 of the alternative dispenser 400 will turn red to indicate that there is no WiFi information saved in the alternative dispenser's flash memory. The user installs and runs the mobile application 606 onto their smartphone 150. On launching, the app 606 causes an “add device” wizard to appear on the screen of the smartphone 150. While following the instructions in the wizard, the user will be presented with a screen to input the network information (i.e network name and password) of their router 604.

The app 606 will connect to the alternative dispenser 400 over a Bluetooth low energy connection (or via WiFi connection initially), and upload the network information to the alternative dispenser 400. The alternative dispenser 400 will then attempt to connect to the router 604. A success or error response will be returned to the smartphone 150 via Bluetooth or WiFi, and displayed to the user on the screen of the smartphone 150.

Once connected to the router 604, the alternative dispenser 400 will attempt to connect to a remote server in the back end 602 over a Message Queue Telemetry Transport (MQTT) connection. The communication between alternative dispenser 400, back end 602 remote server, and smartphone 150 uses a pub/sub model in which all parties can subscribe to topics, and post messages to them. All parties listening in on those topics will receive the messages. Once connected to the MQTT server, the alternative dispenser 400 will remain idle until the smartphone 150 has established a connection to the MQTT server.

Once the wizard is complete on the smartphone 150, a main screen for the app 606 will appear on the smartphone 150. In the background, the app 606 is attempting a connection to the MQTT server. Once connected, the app 606 sends a ‘status request’ packet to a certain channel composed of the mac address for the alternative dispenser 400 and the word ‘control’. The alternative dispenser 400 (which has subscribed to that channel earlier) will receive this message and report back the presence of electrical contact with the left and right fragrance cartridges 48, as detected by the circuit board 92, and optionally other status factors such as whether the front cover 24 is present. The user interface of the app 606 will show visual cues on the smartphone 150 to indicate that the fragrance cartridges 48 are present or removed from the alternative dispenser 400.

The alternative dispenser will remain in a tight loop reporting various parameters such as changes in the front cover 24, presence of the left and right fragrance cartridges 48, motion detector 408 voltage or voltage relative to certain reference values, and whether the burst button 410 is being pressed. The device will also listen for messages from the smartphone app 606, such as commands to emit a burst from the left fragrance cartridge 48 or the right fragrance cartridge 48.

When the alternative dispenser 400 receives a command to emit burst of fragrance from a fragrance dispenser 400, the circuit board 96 turns connects power to the piezoelectric atomizer in the fragrance cartridge 48 and begins a timer. Once a predetermined amount of time, for example 0.1 to 1.0 seconds, has elapsed, power is disconnected from the piezoelectric atomizer in the fragrance cartridge 48.

The user, through their smartphone 150 and a user interface of the app 606, can also set schedules for when the user wishes either of the fragrance cartridges 48 to emit a burs of fragrance. Once a schedule is entered into the app 600, the schedule is serialized into a packet and sent over a MQTT channel, via the back end 602 server. The alternative dispenser 400 will receive the packet, decode it, and store the schedule to its non-volatile flash memory to be executed at the appropriate time

Since communication occurs over the back end 602 MQTT server, the back end 602 server may store all information in the communications for example for analytical and troubleshooting purposes.

The alternative dispenser 400 also subscribes to a common Over The Air (OTA) update channel. When a firmware update is available, the back end 602 server will send out a message to all devices listening on that channel that a firmware update is available. The packet will contain a version number, and file name. The alternative dispenser 400 will compare the version number to its own firmware. If the version number in the message is higher than the current version, the alternative dispenser 400 will perform a request to download the new firmware file from the back end 602 server. The file will be downloaded via an HTTP GET request. The alternative dispenser will then validate the firmware using a Cyclic Redundancy Check (CRC). If the CRC passes, the firmware upgrade process will start, and the alternative dispenser 400 will then reboot.

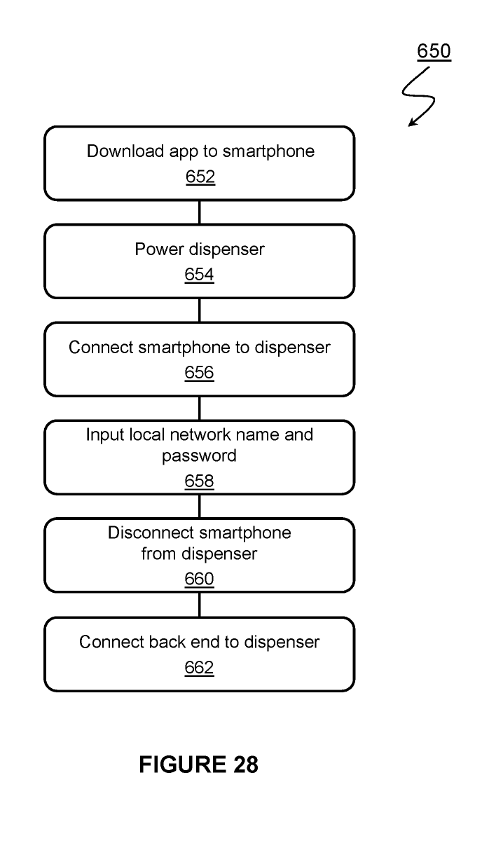

The further description below will elaborate on the description above or give examples of additional or alternative processes in which the elements of the alternative system 600 interact with each other or the user using the alternative system 600 described above. In FIGS. 28 to 34 various processes are described as a sequence of steps. However, the sequential description is not meant to suggest that the steps must necessary be performed in the order shown in the Figures.

FIG. 28 shows a process for initial set up 650 of the alternative dispenser 400 and smartphone 150. The user downloads 652 the app 606 to their smartphone 150 through the website 614. Optionally, the app 606 may provide instructions to the user for the remainder of the initial set up 650, or these instructions may be provided on printed sheets included with the alternative dispenser 400 or through the website 614. The user powers 654 the alternative dispenser 400 by plugging its USB cable 140 or power adapter 36 into a source of power. The user connects 656 the smartphone 150 to the alternative dispenser 400 through the router 604 or directly, for example by Bluetooth communication. The user then inputs 658 the local network name and password to be used by the back end 602 to communicate with the alternative dispenser 400. The user may also input the location of the alternative dispenser and a customer identifier, which is optionally generated without reference to any actual personal information of the user. This optional information may be used to help manage multiple alternative dispensers 400 owned by a single user but placed in different rooms. The user then disconnects 660 the smartphone 150 from the alternative dispenser 400. The back end 602 then connects 662 to the alternative dispenser 400. The alternative dispenser 400 and the back end 602 are now in communication with each other through the router 604.

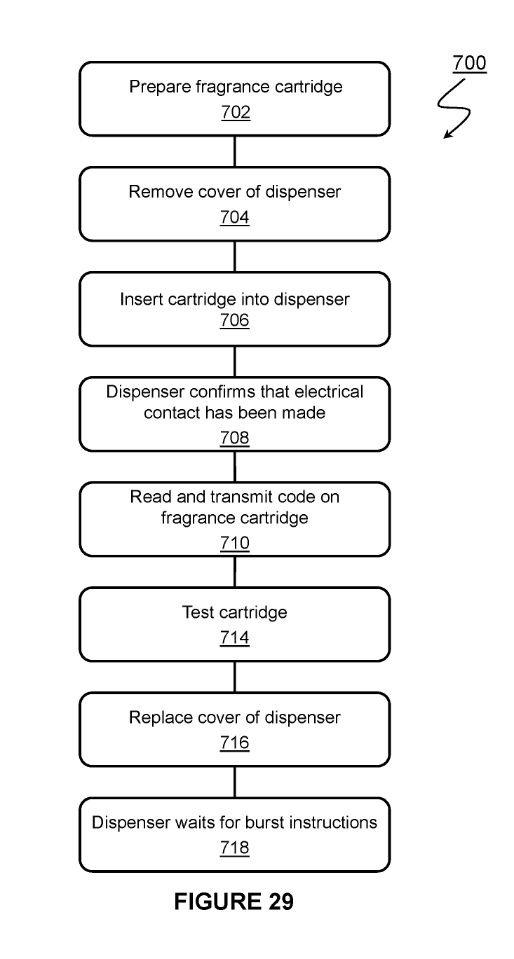

FIG. 29 shows a process to load 700 a new fragrance reservoir 52, 430 into the alternative dispenser. The user prepares 702 the fragrance cartridge 48 by unscrewing a cap from the fragrance reservoir 52, 430, removing any other seal on the fragrance reservoir 52, 430, and screwing the atomizer assembly 56 to the fragrance reservoir 52, 430. The user removes 704 the front cover 24 of the alternative dispenser 400. The user inserts 708 the fragrance cartridge 48 into the alternative dispenser 400. Optionally, the alternative dispenser 400 then confirms 708 that electrical contact has been made with the fragrance cartridge 48, for example by flashing a light or sending a message to the smartphone 150. A code on the fragrance reservoir 52, 430 is then read and transmitted 710 to the back end 602. The code may be provided, for example, with a printed QR code label attached to the fragrance reservoir 52, 430 or provided on or in a box that the fragrance cartridge 52, 430 is shipped with. The QR code is read through a camera on the smartphone 150 and transmitted by the smartphone 150 to the back end 602 with an indication, entered by the user through the touchpad of their smartphone 150, of whether the fragrance reservoir 52, 430 has been loaded into the left or right side of the alternative dispenser 400. In another example, the fragrance reservoir 52, 430 has an RFID tag that is read by an RFID tag reader provided on the circuit board 96 of the alternative dispenser 400. The alternative dispenser 400 transmits the code to the back end 602. The code contains a unique identifier for each individual fragrance reservoir 52, 430 and optionally other information, for example the name of the fragrance in the fragrance reservoir 52, 430. The back end 602 checks that the code is on a list of valid codes (i.e. codes applied during the manufacture of fragrance reservoirs 52, 430 filled with properly formulated fragrance) before allowing the fragrance dispenser 400 to emit bursts from the fragrance reservoir 52, 430. The back end 602 may maintain a database linking the code with information such as fragrance type, time of installation and number of bursts emitted. Optionally, the user can test 714 the fragrance cartridge 48 by pushing a button marked test that appears on their smartphone 150 when a fragrance cartridge 48 is installed. Pushing the test button may also cause the alternative dispenser 400 to make a large burst to both test the fragrance cartridge 48 and prime the atomizer assembly 56, for example by wetting the atomizer disc 84 with fragrance from the new fragrance reservoir 52, 430. The user replaces 716 the front cover 24 of the alternative dispenser 400. The alternative dispenser 400 then waits 718, for example in stand-by, sleep or off mode, for further instructions to emit bursts of fragrance.

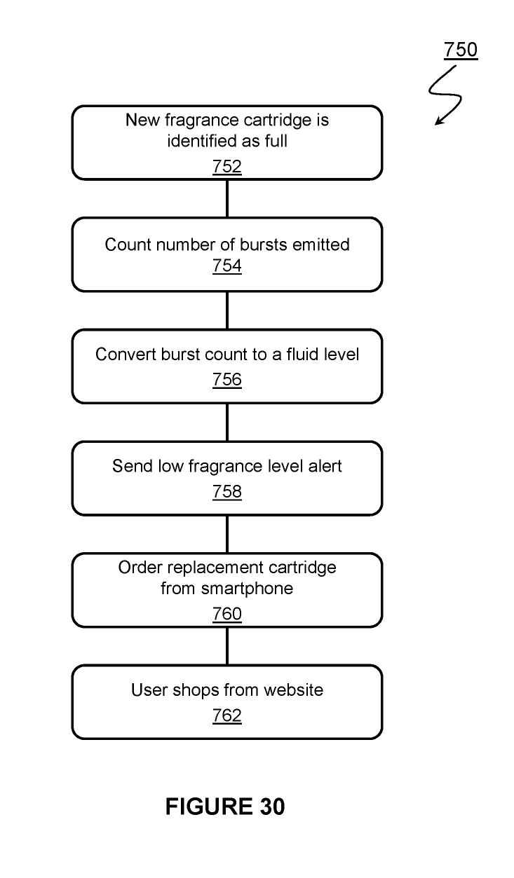

FIG. 30 shows a method of managing fragrance use 750. After a new fragrance reservoir 52, 430 is loaded 700 as described above, it is identified 752 as full in a database maintained by the back end 602. As the alternative dispenser 400 is used, the number of bursts emitted from the fragrance reservoir 52, 430 is counted 754. To allow the count to be maintained, the alternative dispenser 400 may send a message to the back end 602 every time it makes a burst, or the back end 602 may track instructions given to the alternative dispenser 400 through the smartphone 150 or a burst button on the alternative dispenser 400, or the alternative dispenser 400 may maintain a count in its flash memory. The count is maintained either as a forward count starting from 0 to a predetermined maximum number of bursts that can be emitted from a fragrance reservoir 52, 430 or as a count down starting from the predetermined maximum number of bursts that can be emitted from a fragrance reservoir 52, 430. The burst count is converted 756 to an estimated fluid level. This conversion is done automatically as the count is updated. The conversion is expressed, for example as a percentage of fragrance used or remaining, on the smartphone 150 while the user has the app 606 open, or as an alert sent from time to time from the back end 602 or alternative dispenser 400 to the smartphone 150.

In another option relating to the methods of FIGS. 29 and 30, a radio frequency identification (RFID) tag is attached to the fragrance reservoir 52, 430 and programmed with information describing the fragrance in the fragrance reservoir 52, 430, optionally a version number of the fragrance reservoir 52, 430 that can be correlated with, or is, its maximum number of bursts, and an encrypted serial number. When a cartridge 48 is installed into the alternative dispenser 400, the circuit board 96 polls the RFID tag, receives and decrypts the serial number, and sends the serial number to the back end 602 for verification. If the serial number is recognized by back end 602 as representing a usable full fragrance reservoir 52, 430, the back end 602 instructs the alternative dispenser to enter a bursting mode for its left or right side, corresponding to where the verified fragrance reservoir 52, 430 is installed. In the bursting mode, the alternative dispenser 400 will act on instructions to emit bursts of fragrance from its relevant side. The alternative dispenser 400 maintains a counter within its flash memory to track the number of bursts emitted from the fragrance reservoir 52, 430. Once the number of bursts is reached, the alternative dispenser transitions itself to a non-bursting mode on its relevant side and will no longer emit bursts from that fragrance reservoir 52, 430.

Once the maximum number of bursts has been reached, the back end 602 will cancel all future instructions to emit bursts, or instruct the alternative dispenser 400 to enter a non-bursting mode, or the alternative dispenser 400 will put itself into a non-bursting mode. This prevents further bursts from being emitted from the fragrance reservoir 52, 430 linked to the count. The fragrance reservoir 52, 430 is discarded. Further bursts are permitted only after a code from a new replacement fragrance reservoir 52, 430 is provided as the new fragrance reservoir 52, 430 is loaded 700. The back end 602 will only validate a fragrance dispenser 52, 430 once such that a fragrance dispenser cannot be used after it is deemed empty. Once the number of bursts remaining reaches a predetermined level, the back end 602 sends 758 a low fragrance level alert to the smartphone 150. The alert, once received by the smartphone 150 and processed by the app 606, provides a notice on the screen of the smartphone 150 telling the user that the fragrance level is low. Optionally, the alert message may be displayed continuously or periodically while the user continues to emit bursts from the fragrance cartridge 48. The app also provides the user with a virtual button displayed on the screen of the smartphone that, when pushed, orders 760 a replacement fragrance reservoir 52, 430 from the smartphone by sending a message to the back end 602. The back end 602 processes the ordering message according to a replacement cartridge protocol pre-entered by the user. Optionally, the user can shop 762 for one or more fragrance reservoirs 52, 430 from the website 614, which may contain a larger selection of fragrances, payment or delivery options.

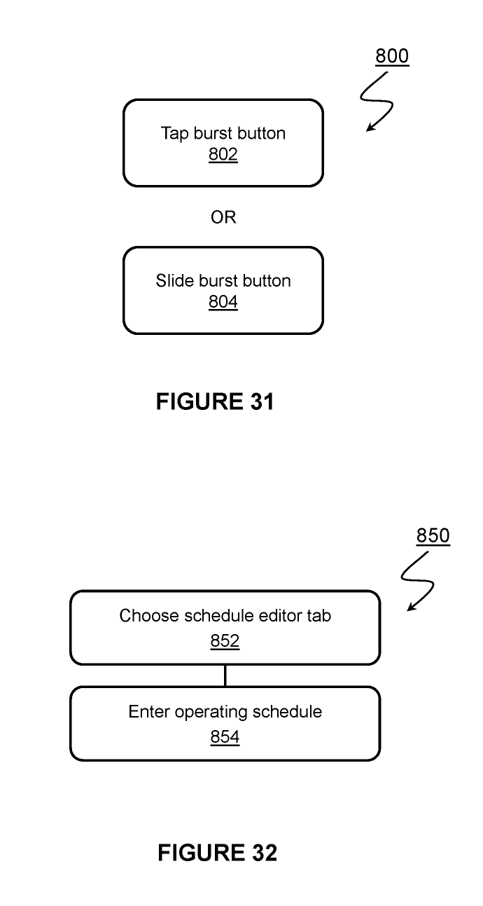

FIG. 31 shows a method for emitting a single burst using the smartphone 800. The user opens the app 606 on their smartphone 150. The app 606 displays, either immediately or after moving through one or more screens, an instruction screen for a particular alternative dispenser 400. The instruction screen may display, for example, the name (i.e. location) of the alternative dispenser 400, an indication of the fragrance type in the left and right fragrance cartridges 48, an indication of the estimated fill level of each fragrance reservoirs 52, 430, a button to close the app 606 or move to a previous screen, buttons to tab into screens for configuring the manual, motion activated or scheduled operation of the alternative dispenser 400, and buttons to provide a burst from the left or right side fragrance cartridges 48. Buttons may be provided by a display indicated a physical button to be pushed to provide the desired function or by a virtual button in the form of an area of the smartphone 150 touchscreen that will be recognized as a button by the app 606. The user may tap 802 a burst button, which causes the app 606 to send a command to the alternative dispenser 400 through back end 602 to emit one burst from the associated fragrance cartridge 48. Alternatively, the user may slide 804 the burst button. This causes the app 606 to send instructions for a sequence of bursts that may have been entered by the user into the app 606 or be a default sequence pre-programmed into the app 606.

FIG. 32 shows a method of programming scheduled operation 850 of the alternative dispenser 850. On the smartphone 150, from the instruction screen described above, the user selects a schedule editor tab 852. This opens up one or more other screens through which the user can select an operating schedule 854. The operating schedule can include, for example, start and end times of operation to be repeated on a daily or weekly schedule. Within a selected time period, for example an hour or more, the alternative dispenser 400 can emits at a preselected frequency, or at a frequency entered by the user when selecting the schedule. The bursts may be distributed evenly over time or varied over time to account for, for example, build up of fragrance in the room or desensitization of the sense of smell. Once confirmed, the schedule is transmitted to the back end 602, which sends burst commands to the alternative dispenser according to the schedule.

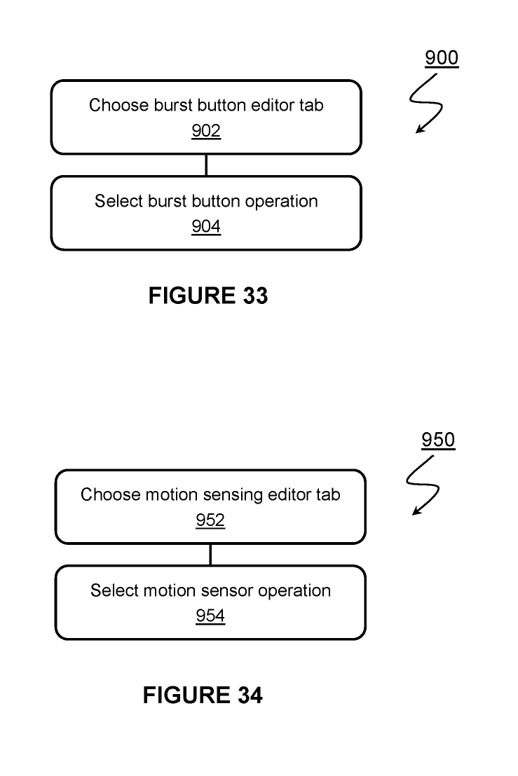

FIG. 33 shows a process for configuring a burst button 900 located on the alternative dispenser 400. On the smartphone 150, from the instruction screen described above, the user selects a burst button editor tab 902. This opens up one or more other screens through which the user can select the burst button operation 904. In one example, the user selects whether the left side or rights side fragrance cartridge 48 will give a burst, and optionally whether it will give a single burst or bursts over a period of time. In another example, the user can select a sequence of touches that will activate either the left or right side fragrance dispenser 48, for example one touch to give a burst form the left side and one touch to give a burst from the right side. A user typically puts a different type of fragrance in the left and right side of the alternative dispenser 400 such that differentiating between the left and right side fragrance dispenser 48 also causes differentiation in fragrance. Once confirmed, the selections are transmitted to the back end 602. The back end 602 may then configure firmware in the circuit board 96 of the alternative dispenser 400 to act according to the selections.

FIG. 34 shows a process for configuring a motion sensor 952 located on the alternative dispenser 400. On the smartphone 150, from the instruction screen described above, the user selects a motion sensor editor tab 952. This opens up one or more other screens through which the user can select the motion sensor operation 954. In one example, the user selects whether the left side or rights side fragrance cartridge 48 will give a burst when the motion sensor detects a person, and optionally whether it will give a single burst or bursts over a period of time. Once confirmed, the selections are transmitted to the back end 602. The back end 602 may then configure firmware in the circuit board 96 of the alternative dispenser 400 to act according to the selections.

While, in the system 146 and alternative system 600 described above, the mobile device or user's computer is a smartphone 150, other types of computers or mobile devices can be employed. For example, networked tablets, personal digital assistants, programmable media remotes, and network-enabled watches can be used to control the fragrance dispenser. Further, other types of non-mobile computing devices, such as desktop computers, web-enabled consoles, etc., can also be employed. The fragrance dispenser may also be controllable via home control platforms like Apple Homekit™ and IBM X10™.

Although the invention has been described with reference to certain specific embodiments, various modifications thereof will be apparent to those skilled in the art. For example, but without limitation, any one or more elements or steps described in relation to one example, embodiment or Figure, can be combined with one or more elements or steps described in relation to one or more other examples embodiments or Figures. The scope of the claims should not be limited by the preferred embodiments, but should be given the broadest interpretation consistent with the description as a whole.

1. A fragrance dispenser comprising a fragrance reservoir disposed in fluid communication upon an atomizer assembly, the atomizer assembly having an atomizer to permit emitting of liquid fragrance communicated from the fragrance reservoir toward the atomizer by gravity when the fragrance dispenser is in an operating orientation.

2. The fragrance dispenser of claim 1 wherein the atomizer comprises a porous plate with a slanting orientation, for example at between 30 and 60 degrees from horizontal.

3. The fragrance dispenser of claim 1 wherein the fragrance reservoir and atomizer assembly form a cavity that is sealed except for a porous plate of the atomizer assembly.

4. The fragrance dispenser of claim 1 wherein the atomizer assembly comprises a piezoelectric atomizer with a porous plate.

5. (canceled)

6. The fragrance dispenser of claim 1 further comprising a button connected to a circuit board configured to activate a piezoelectric atomizer of the fragrance dispenser when the button is touched.

7. The fragrance dispenser of claim 1 further comprising a motion sensor.

8. (canceled)

9. (canceled)

10. (canceled)

11. The fragrance dispenser of claim 1 wherein the fragrance reservoir contains a mixture comprising one or more essential oils and a carrier.

12. (canceled)

13. (canceled)

14. (canceled)

15. A system comprising,
an electrically powered fragrance dispenser adapted to receive messages through a communications network; and,
an application program executable on a personal computer adapted to send messages through the communications network to the fragrance dispenser, or a remote server, or both.

16. The system of claim 15 wherein the fragrance dispenser further comprises a touch activated button.

17. The system of claim 15 further comprising a remote computer in communication with the personal computer and the fragrance dispenser.

18. The system of claim 15 wherein the fragrance dispenser comprises a circuit board configured to enable WiFi communication with the fragrance dispenser.

19. The system of claim 15 wherein the remote computer comprises data storage including a record of the amount of fragrance released from the fragrance dispenser.

20. The system of claim 19 wherein the data includes a count of the number of bursts of fragrance emitted by the fragrance dispenser.

21. The system of claim 18 wherein the count is maintained for a specific replaceable fragrance reservoir of the fragrance dispenser.

22. A method of operating an electrically actuated fragrance dispenser comprising steps of,
connecting the fragrance dispenser to a communications network; and,
installing an application program on a computer; and,
instructing the fragrance dispenser to operate via the computer and the communications network.

23. The method of claim 22 further comprising routing the instructions through a remote computer.

24. The method of claim 23 further comprising providing a replaceable fragrance reservoir with a unique identifier and storing data relating to an amount of fragrance dispensed from the replaceable fragrance reservoir.

25. The method of claim 24 further comprising preventing further release of fragrance from the replaceable fragrance reservoir when it is estimated to be empty.

26. The method of claim 24 further comprising verifying the unique identifier before allowing fragrance to be dispensed from the replaceable fragrance reservoir.

27. The method of claim 22 comprising preparing a schedule for emitting fragrance on the application program and uploading the release schedule to the dispenser.

28. The method of claim 22 comprising sending a burst command from the computer to the dispenser.
